(19) United States
(12) Patent Application Publication
(10) Pub. No.: US 2018/0002692 A1
(43) Pub. Date: Jan. 4, 2018

(54) Sequence Specific and Organism Specific Antimicrobials and Related Materials and Methods

(71) Applicants: Anushree CHATTERJEE, Lafayette, CO (US); Colleen COURTNEY, Boulder, CO (US); The Regents of the University of Colorado, a body corporate, Denver, CO (US)

(72) Inventors: Anushree Chatterjee, Lafayette, CO (US); Colleen Courtney, Frederick, CO (US)

(21) Appl. No.: 15/546,971

(22) PCT Filed: Feb. 1, 2016
(86) PCT No.: PCT/US16/16017

§ 371 Date: Jul. 27, 2017

Related U.S. Application Data
(60) Provisional application No. 62/109,799, filed on Jan. 30, 2015.

(51) Int. Cl. C12N 15/113; C12Q 1/68; A61K 31/713; A61K 45/06

(52) U.S. Cl. CPC C12N 15/113; A61K 45/06; C12N 15/1137; C12Q 1/6876; C12Y 599/01003; A61K 31/713; C12N 2310/11; C12N 2310/3513; C12Q 2600/158; C12Q 2600/178; C12N 2320/31

(57) ABSTRACT

Described herein are RNA-based antisense therapeutics to target antibiotic resistant bacteria. The antisense therapeutics disclosed herein can be useful in re-sensitizing drug-resistant bacteria to antibiotics, as well as developing antibiotics that have bactericidal or bacteriostatic effects on the drug-resistant bacteria or are capable of preventing emergence of antibiotic resistance. Also described herein are methods for re-sensitizing a subject to one or more antibiotics in need thereof, methods for identifying target genes involved in adaptive antibiotic resistance in bacteria, and methods for developing antibacterial antisense therapeutics.

### CROSS-REFERENCE TO RELATED APPLICATIONS

This application claims priority to U.S. Provisional Application No. 62/109,799, filed on Jan. 30, 2015, the entire disclosure of which is expressly incorporated herein by reference for all purposes.

### STATEMENT REGARDING FEDERALLY SPONSORED RESEARCH

This invention was not made with any government support.

### SEQUENCE LISTING

The instant application contains a Sequence Listing which has been submitted via EFS-web and is hereby incorporated by reference in its entirety. The ASCII copy, created on Feb. 1, 2016, is named CU03WOU1_SL.txt, and is 13,252 bytes in size.

### BACKGROUND OF THE INVENTION

Antibiotic resistance is one of the world's most pressing health problems (1). Drug-resistant bacteria infect more than 2 million Americans every year, and are responsible for 23,000 deaths annually in the United States alone. Increasing rates of antibiotic-resistant bacterial infections observed in clinical settings is a result of the misuse and overuse of antibiotics prescribed in veterinary and human medicine. This high volume is largely due to inappropriate prescribing of antibiotics. This is problematic, as use of antibiotics can increase selective pressure in a population of bacteria, resulting in the survival of drug resistant bacteria. These selective pressures and a resulting drug resistant bacterium can result from a single regimen of antibiotics.

While antibiotic resistance continues to be a major global public health concern, antibiotic development continues to stagnate; drug screens for new antibiotics tend to rediscover the same lead compounds, antibiotics represent a relatively poor return on research and development investment compared to other classes of drugs, and antibiotic approval through the U.S. FDA has become confusing and generally infeasible over the past decade. The number of new drugs to replace antibiotics rendered ineffective due to the emergence of antibiotic resistant bacteria is not adequate to meet demand. It remains to be seen whether efforts such as the 2012 Generating Antibiotics Incentives Now Act (GAIN Act) and the FDA's Antibacterial Drug Development Task Force will be successful in bringing new antibiotics to market.

As an example, the class of β-lactam antibiotics—including cephalosporins, penicillins, carbapenems, and monobactams—are some of the most frequently prescribed antibiotics for the treatment of bacterial infections. However, the emergence of β-lactamases has caused extensive resistance against them (6). There have been over 1,300 unique β-lactamases identified in clinical settings, demonstrating the evolutionary robustness of this resistance conferring enzyme (7). Due to the onset of resistance from β-lactamases, β-lactam antibiotics are often combined with β-lactamase inhibitors, including clavulanic acid, sulbactam, and tazobactam in therapeutic applications (7-10). Recently, resistance has also developed to the β-lactam/β-lactamase inhibitor combinations due to the emergence of extended spectrum β-lactamases such as carbapenemases (11) and New Delhi metallo-β-lactamase 1 (NDM-1)(12), providing yet another avenue for widespread antibiotic resistance.

Along with inherent drug-resistance of bacteria, resistance to antibiotics can either be acquired via horizontal gene transfer (13) or be facilitated by adaptive resistance due to increased mutagenesis in bacteria under stress conditions (14). For example, Escherichia coli has been shown to increase mutagenesis by 10²-10³ fold in the presence of a stressor (15). Other antibiotic resistance mechanisms can occur, which do not necessarily arise by mutation, including increased hydrolysis, acetylation, glycosylation, efflux pump expression, and altered targets for the antibiotic (16).

### SUMMARY OF THE INVENTION

Described herein are RNA-based antisense therapeutics to target antibiotic resistant bacteria. The antisense therapeutics disclosed herein can be useful in re-sensitizing drug-resistant bacteria to antibiotics, as well as developing antibiotics that have bactericidal or bacteriostatic effects on the drug-resistant bacteria or are capable of preventing emergence of antibiotic resistance. Also described herein are methods for re-sensitizing a subject to one or more antibiotics in need thereof, methods for identifying target genes involved in adaptive antibiotic resistance in bacteria, and methods for developing antibacterial antisense therapeutics.

In one embodiment described herein is an antisense antibiotic oligomer comprising a nucleic acid sequence complementary to at least one target selected from the group of: at least one target site on a DNA sequence of an essential bacterial gene associated with an antibiotic pathway; at least one target site on a DNA sequence of a bacterium associated with antibiotic resistance; at least one target site on an RNA sequence of the bacterium associated with antibiotic resistance; at least one target site on an mRNA sequence of the bacterium which encodes a protein essential for bacterial homeostasis; and at least one target site on an mRNA sequence of the bacterium which encodes a protein associated with antibiotic resistance.

In another embodiment provided herein is an antibiotic composition comprising at least one antisense antibiotic oligomer described herein.

In yet another embodiment described herein is a method for treating a bacterial infection in a subject in need thereof, comprising re-sensitizing a subject to one or more conventional antibiotics by administering to the subject a pharmaceutically effective amount of a composition described herein wherein the at least one antisense antibiotic oligomer targets at least one of a DNA sequence, RNA sequence, mRNA sequence associated with antibiotic resistance and administering to the subject at least one antibiotic to which the subject has been re-sensitized.

In yet another embodiment provided herein is a method for treating a bacterial infection in a subject in need thereof, comprising administering to the subject a pharmaceutically effective amount of a composition described herein wherein the at least one antisense antibiotic oligomer is bactericidal or bacteriostatic.

In yet another embodiment described herein is a method for preventing emergence of antibiotic resistance in a subject in need thereof, comprising administering to the subject a pharmaceutically effective amount of a composition described herein wherein the at least one antisense antibiotic oligomer targets at least one of a DNA sequence, RNA sequence, or mRNA sequence associated with antibiotic resistance.

In yet another embodiment provided herein is a method for identifying target genes involved in adaptive antibiotic resistance in bacteria comprising: pretreating antibiotic-resistant bacteria with at least one antisense antibiotic oligomer described herein; incubating the pretreated antibiotic-resistant bacteria with the at least one antisense antibiotic oligomer and an antibiotic to which the bacteria is resistant; selecting one or more colonies of appearing after the incubation step; determining the expression of two or more stress response genes; determining a fold increase for each of the two or more stress response genes relative to a control wherein antibiotic-resistant bacteria were not pretreated with the composition; and identifying a target gene involved in adaptive antibiotic resistance, wherein a fold increase of about 2 or more for a particular gene as determined in the fold increase determining step is indicative of a target gene involved in adaptive antibiotic resistance.

In yet another embodiment described herein is a method for developing an antibacterial antisense therapeutic comprising: identifying at least one target gene involved in adaptive antibiotic resistance; and designing an antisense oligomer complimentary to an mRNA sequence of the at least one target gene identified in step a), thereby developing an antibacterial antisense therapeutic.

### BRIEF DESCRIPTION OF THE DRAWINGS

The patent or application file contains one or more drawings executed in color and/or one or more photographs. Copies of this patent or patent application publication with color drawing(s) and/or photograph(s) will be provided by the Patent Office upon request and payment of the necessary fee.

FIGS. 1A-1B: Schematic of RNA inhibitor activity against TEM-1 β-lactam resistant bacteria. FIG. 1A) Diagram showing the mechanism for re-sensitizing drug-resistant bacteria to an antibiotic. The RNA inhibitors exhibit translational inhibition and prevent the β-lactamase mRNA transcript from being translated to the β-lactamase enzyme. In absence of RNA inhibitors, drug-resistant bacteria survive and proliferate in presence of the β-lactam antibiotic. In presence of RNA inhibitors, the reduced expression of β-lactamase causes the drug-resistant bacteria to be re-sensitized to β-lactam antibiotics. FIG. 1B) Diagram showing Target sites of PNA-based antisense inhibitors on secondary structure of bla RNA (SEQ ID NOS 50-51) encoding the β-lactamase enzyme (left panel). PNA molecules are 12-mers where the C-terminal is conjugated to an O-linker conjugated to (KFF)₃K (SEQ ID NO:49) cell penetrating peptide. The ribosomal binding site (RBS), translational start site (TSS) and the YUNR motif are underlined in the bla sequence (right panel) (SEQ ID NO: 52). Antisense agents α-RBS (SEQ ID NO: 1), α-TSS (SEQ ID NO: 3), and α-YUNR (SEQ ID NO: 5) targeting ribosomal binding site, translational start site and YUNR motif respectively are shown.

FIG. 2: Minimum inhibitory concentration of RNA inhibitors. Box plot showing normalized growth of overnight cultures for treatment with different concentrations of RNA inhibitors. Specific growth rate of cells under treatment with RNA inhibitors (y-axis) is normalized to specific growth rate in absence of treatment. The cultures are treated with respective concentrations of RNA inhibitors either in absence (empty data points) or presence of ampicillin (filled data points) (300 μg/mL). The minimum inhibitory concentration (MIC) of α-TSS, α-RBS and α-YUNR are shown at 2.5 μM, 25 μM, and greater than 25 μM, respectively. Significance (p<0.05) is represented with an asterisk. Data shown is an average of three independent experiments (error bars are standard deviation from average values).

FIGS. 3A-3B: Mechanism of action of the RNA inhibitors. FIG. 3A) Bar graph showing bla mRNA expression in E. coli treated with respective antisense molecule in the absence of ampicillin No significant change in bla mRNA expression is observed. FIG. 3B) Bar graph showing β-lactamase activity assay with respective antisense oligomer in the absence of ampicillin α-RBS is at 25 μM, α-TSS is 5 μM, and α-YUNR is 25 μM for both (FIGS. 3A-3B). Data shown in FIGS. 3A-3B are an average of three independent experiments (error bars are standard deviation from average values).

FIGS. 4A-4C: Re-sensitization of drug-resistant bacteria using α-TSS. FIG. 4A) Bar graph showing colony forming units/mL (CFU/mL) for cultures treated with different concentrations of α-TSS and 300 μg/mL ampicillin. Overnight cultures were pretreated with respective concentrations of α-TSS, followed by 1:10 dilution in presence of respective of α-TSS concentrations and 300 μg/mL ampicillin. Cultures with significant decrease in growth (p<0.05) are represented with an asterisk. FIG. 4B) Representative colony growth on petri dishes of cultures treated with different concentrations of α-TSS and 300 μg/mL ampicillin. Plates show CFU from 10 μL of 1:100 dilution of culture at t=3 hr. FIG. 4C) Ampicillin sensitivity analysis for cultures treated with 0 μM, 0.5 μM (below MIC) and 5 μM (above MIC) of α-TSS in presence of range of ampicillin concentrations. Optical density (562 nm) of cultures treated with a range of α-TSS and ampicillin concentrations for 24 hr is shown. The three conditions show different degrees of sensitivity as shown using a Boltzman data fit. Data shown in FIGS. 4A and 4C are an average of three independent experiments (error bars are standard deviation from average values).

FIGS. 5A-5C: Mutants adapted to RNA inhibitors demonstrate gene expression heterogeneity. FIG. 5A) Plot showing growth rate for individual replicates in no treatment population (n=6 biological replicates), mutant population 1 (n=6) and mutant population 2 (n=5). FIG. 5B) Box plot showing fold change in expression of representative stress response genes in mutant populations 1 (n=4) and 2 (n=3) with respect to housekeeping gene cysG and no treatment population (n=3). High gene expression variability is observed across mutant populations. FIG. 5C) Hierarchical clustering analysis showing significant difference in coefficient of variation between no treatment case and mutant populations 1 and 2. Heatmap values indicate coefficient of variation of −ΔC_{q,avg} between biological replicates of no treatment population (n=3), mutant population 1 (n=4) and mutant population 2 (n=3). Clustering is based on Euclidean distance. Data shown in FIGS. 5A and 5B are an average of ‘n’ biological replicates (error bars are standard deviation from average values).

FIG. 6: Optimal and suboptimal RNA structures of bla target regions. RNA structures were modeled in RNA structure software. α-RBS (red), α-TSS (green), and α-YUNR (purple) are shown on the sequence with their structure. The “(” and “)” symbols represent nucleotides which are double stranded and “.” symbol represent single stranded nucleotides. The ribosomal binding site (RBS), translation start site (TSS), and YUNR motif for the designed antisense oligomers are indicated. Figure discloses SEQ ID NOS 53-54, respectively, in order of appearance.

FIGS. 7A-7B: Growth curves in the presence of α-TSS. FIG. 7A) Optical density (562 nm) vs. time for three biological replicates in the presence of respective concentrations of α-TSS without ampicillin FIG. 7B) Optical density (562 nm) for three biological replicates which were pretreated overnight in respective concentrations of α-TSS without ampicillin and diluted into respective concentrations of α-TSS and 300 μg/mL ampicillin Data shown are an average of three independent experiments (error bars are standard deviation from average values).

FIGS. 8A-8B: Growth curves in the presence of α-RBS. FIG. 8A) Optical density (562 nm) vs. time for three biological replicates in the presence of respective concentration of α-RBS antisense agent and no ampicillin FIG. 8B) Optical density for three biological replicates which were pretreated overnight in respective concentration of α-RBS without ampicillin and diluted into respective concentration of α-RBS and 300 μg/mL ampicillin. Data shown are an average of three independent experiments (error bars are standard deviation from average values).

FIGS. 9A-9B: Growth curves in the presence of α-YUNR. FIG. 9A) Optical density (562 nm) vs. time for three biological replicates in the presence of respective concentration of α-YUNR antisense agent without ampicillin FIG. 9B) Optical density (562 nm) for three biological replicates which were pretreated overnight in respective concentrations of α-YUNR without ampicillin and diluted into respective concentrations of α-YUNR and 300 μg/mL ampicillin. Data shown are an average of three independent experiments (error bars are standard deviation from average values).

FIGS. 10A-10B: Growth curves of mutant populations 1 and 2. FIG. 10A) Optical density (562 nm) vs. time of mutant population 1 and 2 grown in the presence of 2.5μM α-TSS during their adaptation. FIG. 10B) Optical density (562 nm) vs. time of α-TSS/ampicillin resistant mutant cultures and α-TSS/ampicillin sensitive cultures grown with a no treatment culture in the presence of ampicillin.

FIGS. 11A-11C: Mutant biological replicate and RNA expression. FIG. 11A) Growth curve showing biological replicates grown from freezer stock in 2.5μM α-TSS and 300 μg/mL ampicillin. The mutant samples grew with different growth characteristics including varied lag phase and varied growth rate. The lag phase ranged between 2-12 hr for colonies from mutant population 1 and 7-14 hr for colonies from mutant population 2. The growth rates varied between 0.08-0.14 hr⁻¹ for mutant population 1 and 0.09-0.16 hr⁻¹ for mutant population 2. FIG. 11B) Box plot showing cycle number for the housekeeping gene cysG was consistent across the no treatment population (n=3 biological replicates), mutant population 1(n=4), and mutant population 2 (n=3). Data shown are an average of ‘n’ biological replicates (error bars are standard deviation from average values). FIG. 11C) Plot showing fold change with respect to housekeeping gene cysG for individual biological replicates of the no treatment population, mutant population 1, and mutant population 2.

FIGS. 12A-12B: Design of antisense inhibitors targeting novel essential genes and pathways. FIG. 12A) Pie charts showing current antibiotics and essential genes in E. coli (Keio Collection) broken into ten groups based on function to highlight the disconnect between current antibiotics and available essential target genes. FIG. 12B) Illustration showing the implementation of the novel antisense PNA molecules either as an antibiotic alone, in combination with other antisense molecules to target multiple genes, or in combination with conventional antibiotics for clinical resistant strains.

FIGS. 13A-13E: Antisense antibiotic oligomers targeting novel pathways can inhibit growth of E. coli. FIG. 13A) Time graph showing optical density of MG1655 with 10 μM of respective antisense inhibitor. FIG. 13B) Bar graph showing optical density at 15 h, where no treatment has reached stationary phase, to compare growth with respective treatments. FIG. 13C) Bar graph showing lag time of MG1655 with respective treatments. As shown in FIGS. 13A-13C, α-lexA, α-fnrS and α-rpsD completely prevented cell growth. FIG. 13D) Bar graph showing colony forming units per milliliter for respective treatments as a function of time for MG1655 E. coli. FIG. 13E) Time graph showing normalized colony forming unit analysis where CFU/mL was normalized to individual biological replicate at t=0. The CFU/mL at t=0 represents the starting culture after a 1:100,000 dilution from overnight culture. MG1655 treatment with 10 μM α-rpsD resulted in 0 CFU/mL within 2 hours of treatment. Pound sign (#) represents significant difference from t=0 and asterisk (*) indicates significant difference from no treatment at each specified time point. p<0.05.

FIG. 14: Representation of the severity of drug resistance in the clinical strains used in the present study. These strains are resistant to multiple classes of antibiotics. Knowledge of their resistance was used to focus on combining treatments and re-sensitizing the drug resistant strains with antisense therapeutics as described herein.

FIGS. 15A-15F: Antisense antibiotic oligomers inhibit growth of clinically isolated multi drug resistant bacteria. Time graphs show optical density of respective clinical strains with 10 μM respective treatment in various pathways: FIG. 15A) Metabolism (folC); FIG. 15B) Signaling (ffh); FIG. 15C) Stress response (lexA); FIG. 15D) Small HFQ binding RNA (fnrS); FIG. 15E) Transcription (gyrB); and FIG. 15F) Translation (rpsD).

FIGS. 16A-16F: Antisense antibiotic oligomers inhibit growth of clinically isolated multi drug resistant bacteria. Bar graphs showing optical density at time t where the no treatment condition for each strain reached stationary phase: FIG. 16A) Metabolism (folC); FIG. 16B) Signaling (ffh); FIG. 16C) Stress response (lexA); FIG. 16D) Small HFQ binding RNA (fnrS); FIG. 16E) Transcription (gyrB); and FIG. 16F) Translation (rpsD). Time t was as follows: MDR Salmonella typhimurium (MDR ST) 14 h; carbapenem resistant Enterobacteriaceae Escherichia coli (CRE E. coli) 18 h; MDR E. coli 18 h; extended spectrum β-lactamase producing Klebsiella pneumoniae (ESBL K. pneumoniae) 16 h, and New-Delhi metallo-β-lactamase producing K. pneumoniae (NDM-1 K. pneumoniae) 16 h. Significance from no treatment condition is represented by an asterisk (*) where significance is defined as p<0.05. Data shown are the average of at least three independent biological replicates.

FIGS. 17A-17D: Use of antisense antibiotic oligomers as “resistance-breakers” to re-sensitize multi drug resistant bacteria to antibiotics. FIG. 17A) Schematic of one possible proposed combination of drugs targeting either multiple pathways with multiple targets (left) or the same pathway with the same target (right). This scheme can be used to target both traditional antibiotic targets as well as novel antibiotic targets. Two combinations are shown, one targeting transcription and translation and one which targets transcription with two different therapeutics. FIG. 17B) Time graph showing optical density of CRE E. coli treatment with respective therapeutic mono or combination therapy. FIG. 17C) Bar graph showing optical density and time t=18 h where the no treatment condition for CRE E. coli has reached stationary phase. Asterisk (*) represents significantly different from no treatment, the letter X represents significantly different from antibiotic monotherapy, and the percent sign (%) represents significantly different from antisense PNA monotherapy, where significance is defined as p<0.05.

FIG. 18: Peptide nucleic acid based antisense antibiotic oligomers do not affect mammalian cells. Photograph showing HEK 293T cells after being cultured with 5 μM α-nonsense antisense inhibitor for 24 h demonstrating the benign nature of peptide nucleic acid on mammalian cells. The sequence specificity of antisense therapeutics allows for one to design molecules which do not have targets in mammalian cells.

### DETAILED DESCRIPTION

Described herein are RNA-based antisense therapeutics to target antibiotic resistant bacteria. The antisense therapeutics disclosed herein can be useful in re-sensitizing drug-resistant bacteria to antibiotics, as well as developing antibiotics that have bactericidal or bacteriostatic effects on the drug-resistant bacteria or are capable of preventing emergence of antibiotic resistance. Also described herein are methods for re-sensitizing a subject to one or more antibiotics in need thereof, methods for identifying target genes involved in adaptive antibiotic resistance in bacteria, and methods for developing antibacterial antisense therapeutics.

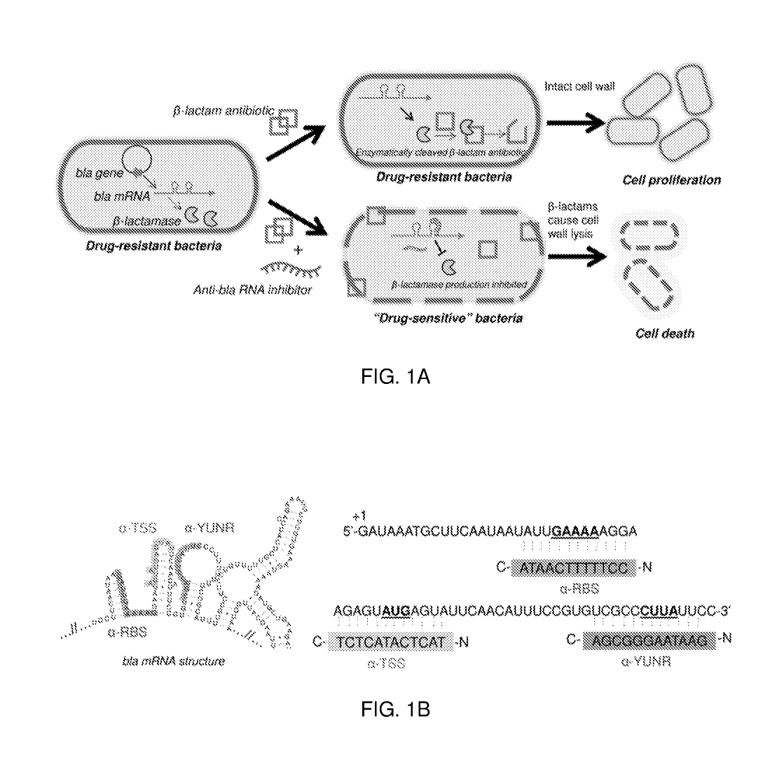

Many of the presently available antibiotics, including β-lactam/β-lactamase inhibitors, have been developed to interfere with bacterial enzymes (17). In particular embodiments, an antisense strategy for targeting β-lactamase gene (bla) mRNA to prevent translation of β-lactamase enzyme is described (FIG. 1A). RNA inhibitors targeting the translation start site and ribosomal binding sites are successful at re-sensitizing drug-resistant E. coli to β-lactam antibiotics. A similar approach can be employed to target other resistance mechanisms such as NDM-1, carbapenemase, aminoglycoside acetyltransferase, and dihydropteroate synthase (35, 36).

In other particular embodiments, an antisense strategy targeting one or more essential genes of non-traditional antibiotic pathways is described. These pathways include one or more of transport and membrane function or biosynthesis, metabolism, redox homeostasis, stress response, cell signaling, replication and growth, transcription and translation, and DNA modifications, repair, and maintenance.

### General Description

Current commercial antibiotics are limited to three main pathways in bacteria: DNA replication and cell growth, protein biosynthesis, and cell wall biosynthesis. Addressing antibiotic resistance is becoming increasingly urgent as serious pathogens are continuing to spread and develop resistance to available antibiotics (1-4). Bacteria are rapidly developing resistance to currently available therapeutics, and fewer therapeutics are being developed (5).

Common drug-resistant bacteria include carbapenem resistant Enterobactericeae Klebsiella pneumonia (CREKP) (2), multidrug-resistant tuberculosis (MDRTB) (3), multi drug resistant Salmonella enterica (4), multi drug resistant Salmonella typhimurium (MDRST), methicillin-resistant Staphylococcus aureus (MRSA), vancomycin-resistant S. aureus (VRSA), extended spectrum β-lactamase Klebsiella pneumoniae (ESBL K. pneumoniae), vancomycin-resistant Enterococcus (VRE), carbapenem resistant Enterobacteriaceae Escherichia coli (CRE E. coli), multi drug resistant Escherichia coli (MDR E. coli), New-Delhi metallo-β-lactamase producing Klebsiella pneumoniae (NDM-1 K. pneumoniae) and multi drug resistant Acinetobacter baumannii (MRAB).

Resistance to β-lactam antibiotics and β-lactamase inhibitors is increasingly complicating the treatment of bacterial infections in the clinical setting. Because the pipeline for novel antibiotics has slowed considerably, it would be beneficial to re-sensitize antibiotic-resistant bacteria to the original antibiotic.

Enzymatic inactivation of an antibiotic is one of the most common mechanisms of antibiotic resistance. And while many inhibitors for these bacterial enzymes have been developed, bacteria continue to develop antibiotic resistance by altering these enzymes, rendering the inhibitors ineffective. Described herein is an antisense-based RNA inhibitor of β-lactamases. It will be recognized by those of skill in the art that a similar approach can be applied to other resistance mechanisms, including but not limited to NDM-1, carbapenemase, aminoglycoside acetyltransferase, and dihydropteroate synthase.

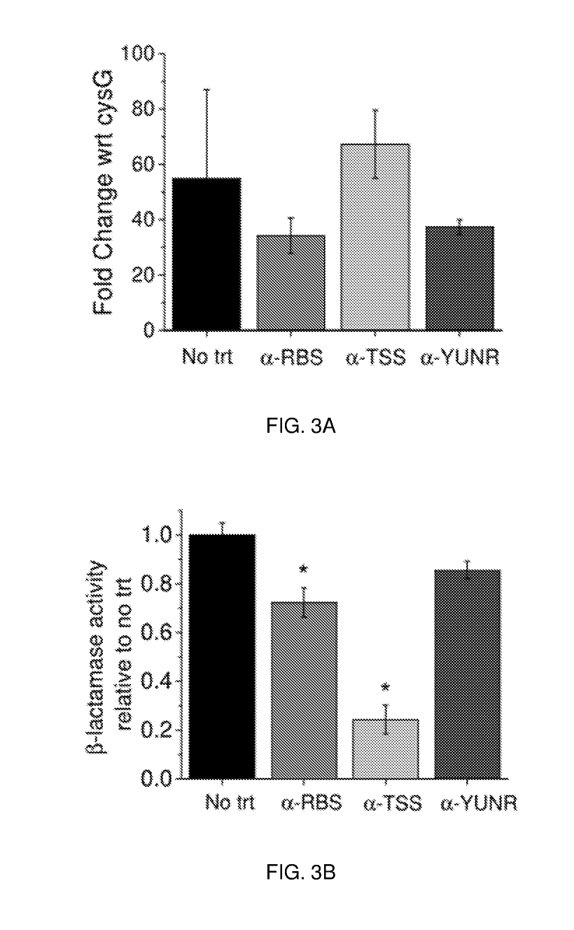

An antisense therapeutic approach was utilized to design antisense antibiotic oligomers against bla mRNA encoding β-lactam resistance. As shown in FIG. 1A, the antisense antibiotic oligomers inhibit β-lactam production, allowing β-lactam antibiotics to cause cell wall lysis resulting in cell death. The mechanism of inhibition of the bla antisense antibiotic oligomers was shown to be inhibition of translation of the encoded β-lactamase (Example III; FIGS. 3A-3B). While RNA expression of bla transcript remained similar both in absence and presence of treatment with the antisense inhibitors (FIG. 3A), two of the antisense inhibitors tested (α-TSS and α-RBS) significantly reduced β-lactamase activity, indicating inhibition of translation (FIG. 3B).

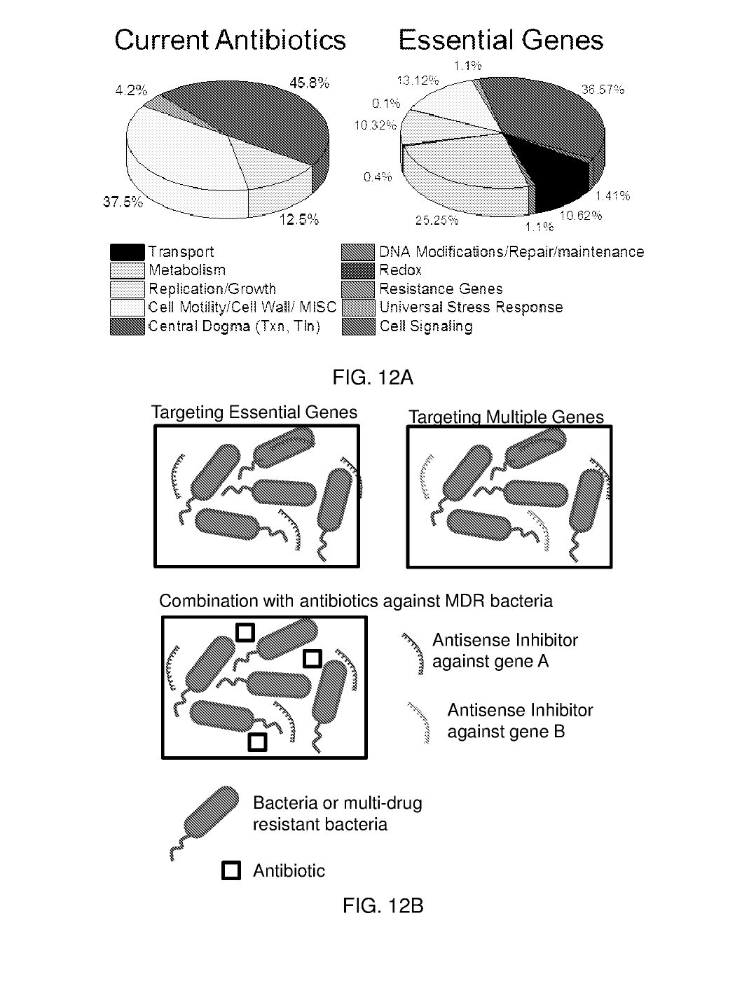

Also described herein are antisense antibiotic oligomers that target essential genes in non-conventional antibiotic pathways. By targeting non-traditional antibiotic pathways, novel antibiotics were generated. As depicted in FIG. 12B, antisense antibiotic oligomers targeting the non-traditional antibiotic pathways can be used on their own, in combination with other antisense antibiotic oligomers, or in combination with conventional antibiotics. Used in combination with other antisense antibiotic oligomers or in combination with conventional antibiotics, multiple antibiotic pathways can be targeted, or multiple sites in one pathway can be targeted. Antibiotic pathways targetable by antisense antibiotic oligomers include but are not limited to transport and membrane function or biosynthesis, metabolism, redox homeostasis, stress response, cell signaling, replication and growth, transcription and translation, and DNA modifications, repair, and maintenance. As shown and described herein, antisense oligomers targeting these pathways can be used as antibiotic therapeutics.

Antisense therapeutics are nucleotide sequence-based therapeutics which target specific RNA or DNA sequences known to be causative of a particular condition or disease. By designing and synthesizing a strand of nucleic acid complementary to the causative RNA or DNA sequence, it is possible to inactivate the causative gene, effectively turning it “off” (FIG. 1A). This is due to the formation of a duplex between the antisense oligonucleotide and a single stranded nucleic acid through complementary Watson-Crick base pairing (see FIG. 1B) (18, 19), which prevents the causative sequence from being translated. Antisense interactions can decrease gene expression by blocking ribosomal binding, preventing ribosomal migration, or inducing cleavage by RNases (20, 21).

Antisense based therapeutics are inherently specific due to their sequence-based targeting. This makes them advantageous as antibiotics because it removes side effects associated with broad-range antibiotics, including preventing extreme changes in the resistome and populations of the subjects' microbiome (37). To date, antisense therapeutics have been mostly applied to target cancer markers in mammalian cells, including targeting telomerase or telomerase reverse transcriptase associated with cancer tumorigenesis (38, 39). Such sequence specificity allows for cancerous cells to be targeted without killing nearby healthy cells. Antisense-based therapies have also been applied to mitigate the effects of inflammatory bowel disease (22), diabetic blindness (23), cardiovascular disease (24), HCV (25), HIV (26), Duchenne muscular dystrophy (40), and heart disease (41).

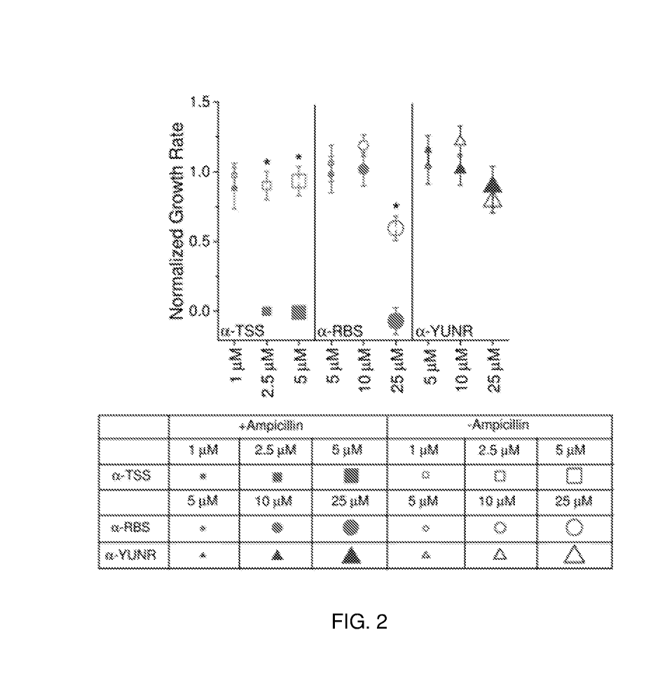

As described in Example 1, antisense antibiotic oligomers targeting the ribosomal binding site (α-RBS) and the translational start site (α-TSS) of bla mRNA caused the re-sensitization of drug resistant E. coli to ampicillin and hindered cell growth in the presence of ampicillin. FIG. 2 shows significant inhibition of growth of overnight cultures of MDR E. coli by 2.5 μM α-TSS in the presence of 300 μg/ml ampicillin and by 25 μM α-RBS in the presence of μg/ml ampicillin. No inhibition was observed in either instance, indicating the non-toxicity of the two antisense antibiotic oligomers.

FIG. 3 shows that antisense antibiotic oligomers α-TSS and α-RBS did not significantly reduce bla expression (FIG. 3A), but rather significantly inhibited β-lactamase activity (FIG. 3B). This indicates that the two antisense antibiotic oligomers function by impairing translation of bla mRNA.

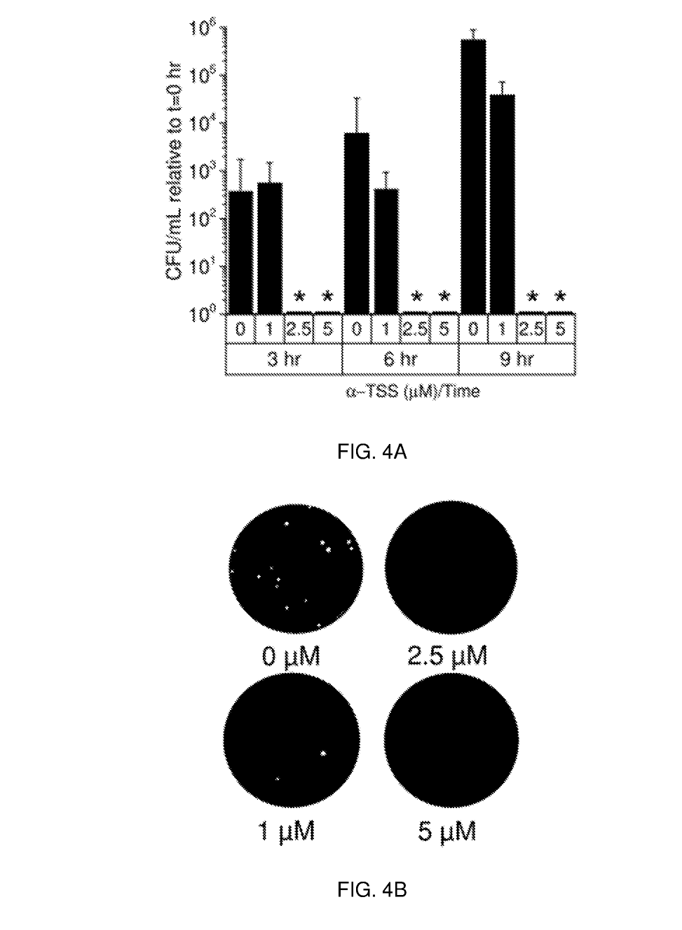

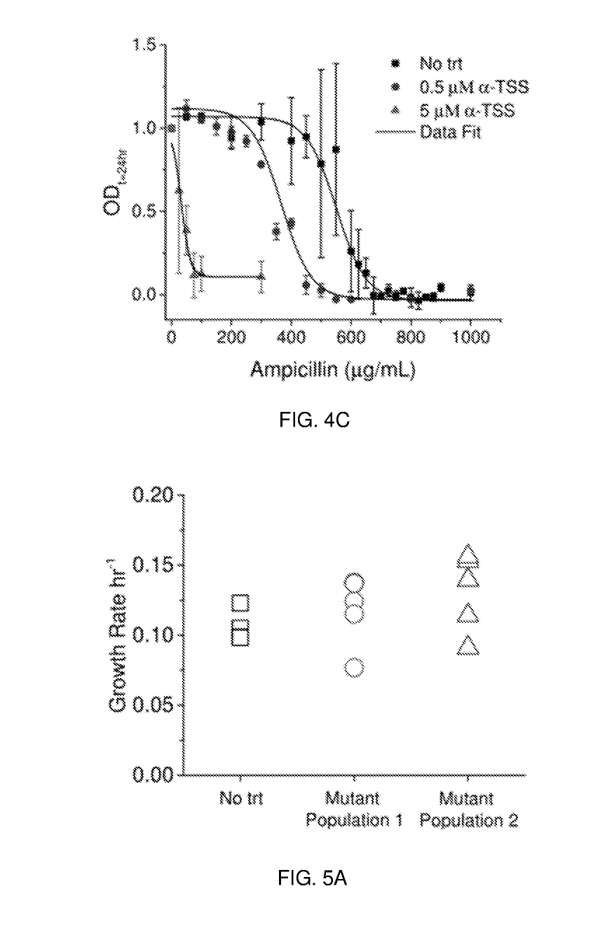

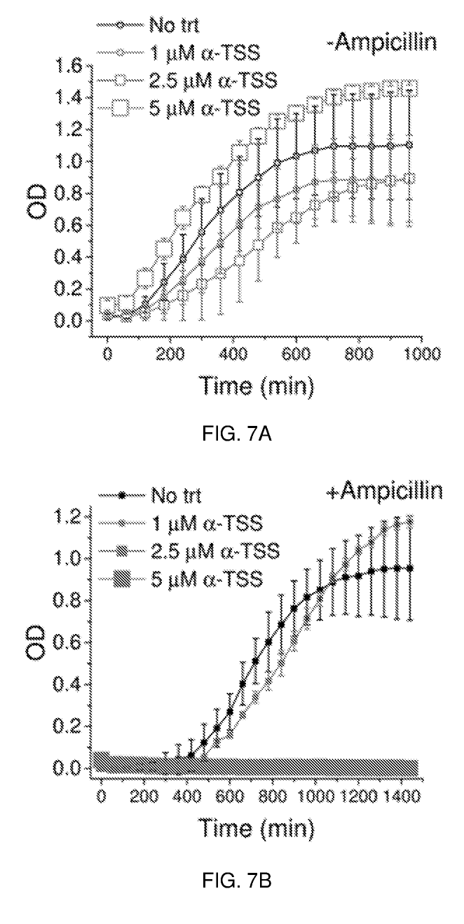

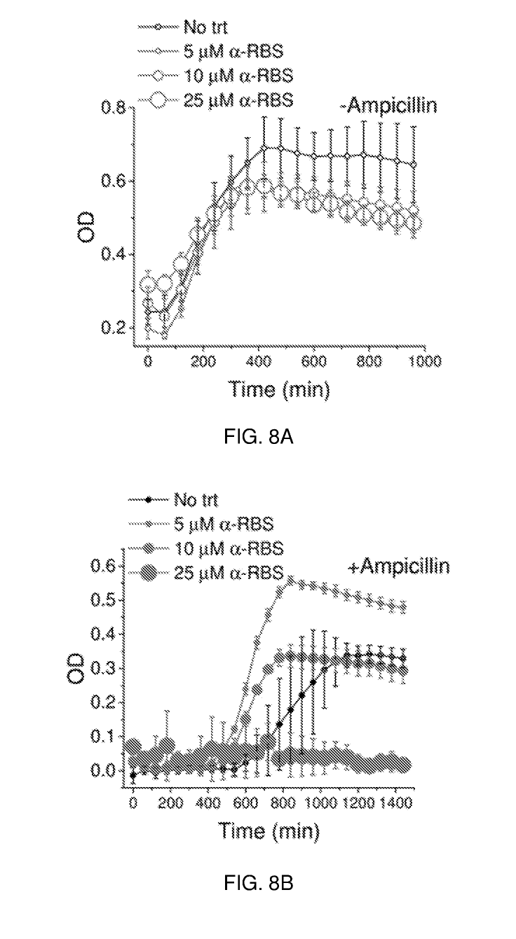

Both α-TSS and α-RBS re-sensitize MDR E. Coli to ampicillin. As shown in FIGS. 7B and 8B, overnight treatment with α-TSS or α-RBS, respectively, followed by treatment with antisense antibiotic oligomer and 300 μg/ml ampicillin resulted in significant antibiotic effect relative to untreated cells (No trt) with the antisense antibiotic oligomer. As depicted by FIG. 4C, treatment of MDR E. coli with α-TSS at concentrations both below its minimum inhibitory concentration (MIC; 0.5 μM) and above its MIC (5 μM) resulted in a leftward shift in ampicillin sensitivity. The increase in sensitivity to ampicillin due to treatment with antisense antibiotic oligomer indicates that such inhibitors can provide for the use of reduced concentrations of conventional antibiotic.

Targeting only about 4 of 10 possible targets, there is a significant disconnect between current antibiotics and available essential genes (FIG. 12A). These essential genes are potential targets of novel antibiotics, including antisense antibiotic oligomers. To further evaluate antisense oligomers as antibiotics, their ability to target non-traditional antibiotic pathways was evaluated. Antisense inhibitors were designed to target E. coli folC, involved in metabolism, ffh, which is a part of the signal recognition particle, lexA, a key regulator of stress response, and fnrS, a small HFq binding RNA. Antisense antibiotic oligomers were also designed against two genes essential to traditional antibiotic pathways in addition to bla; rpsD involved in protein biosynthesis, and gyrB involved in DNA replication. While the antisense inhibitors were designed against E. coli sequences, the inhibitors were designed to also target Klebsiella pneumoniae and Salmonella enterica where possible to create broad-spectrum gene specific antibiotics.

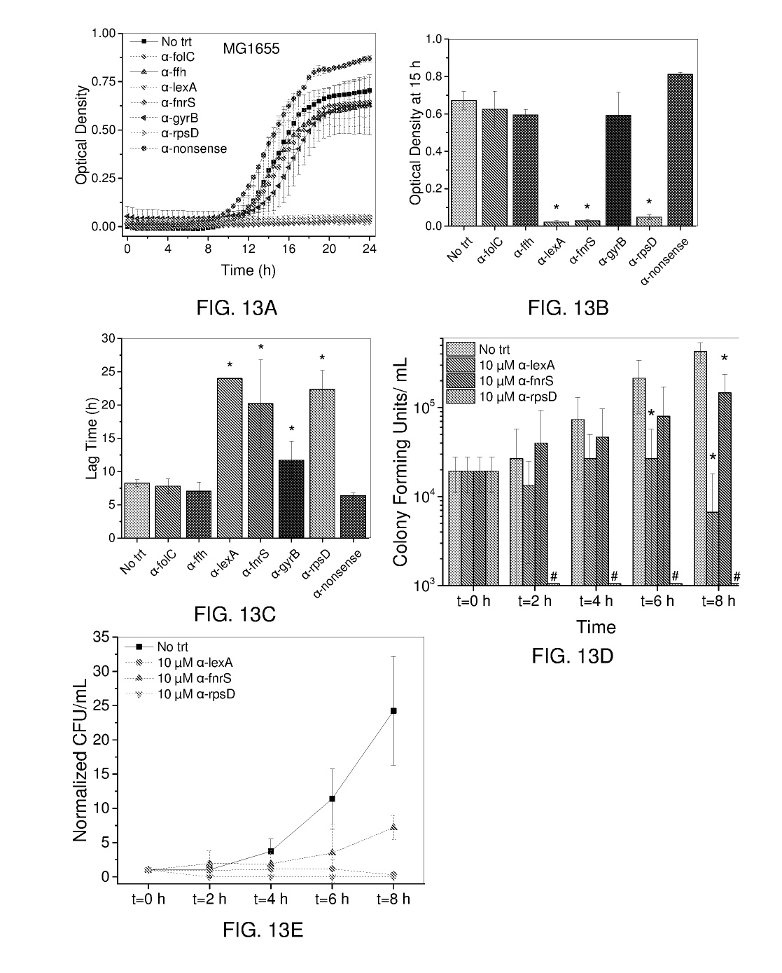

Complete inhibition of MG1655 E. coli growth was achieved for 24 hours in E. coli with 10 μM of antisense inhibitors α-lexA, α-fnrS, and α-rpsD, while treatment with α-lexA, α-fnrS, α-rpsD, or α-gyrB significantly increased lag time (FIGS. 13A-13C). Treatment with 10 μM α-rpsD resulted in the reduction of colony forming unites per milliliter to zero after two hours, indicating the antisense inhibitor's bactericidal effects (FIG. 13D). Antisense inhibitor α-lexA prevented significant increase in viable cells at six and eight hours, demonstrating the inhibitor's bacteriostatic effect on cell growth. In conjunction with the bla results, these data demonstrate how antisense inhibitors targeting traditional and non-traditional antibiotic pathways can be effectively used as antibiotics.

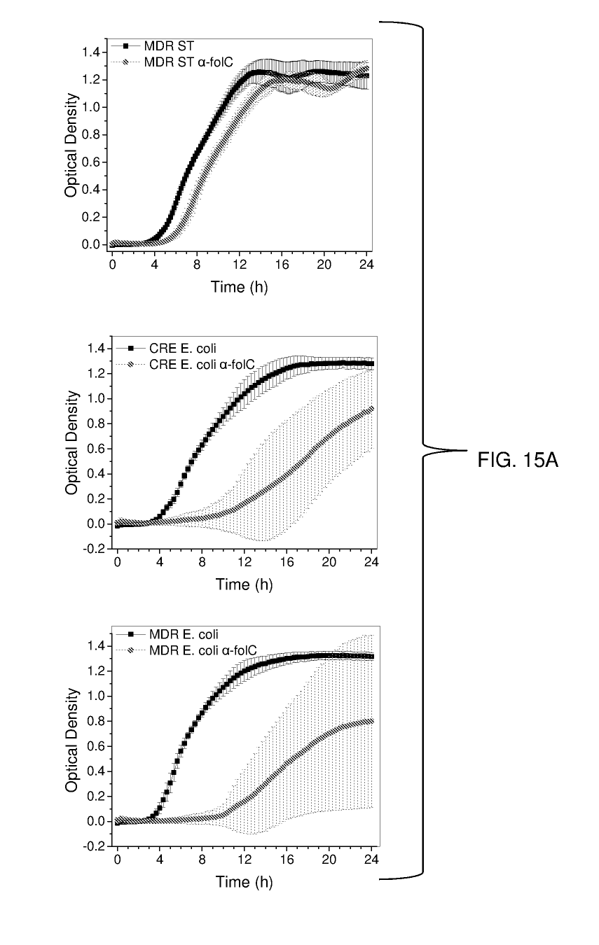

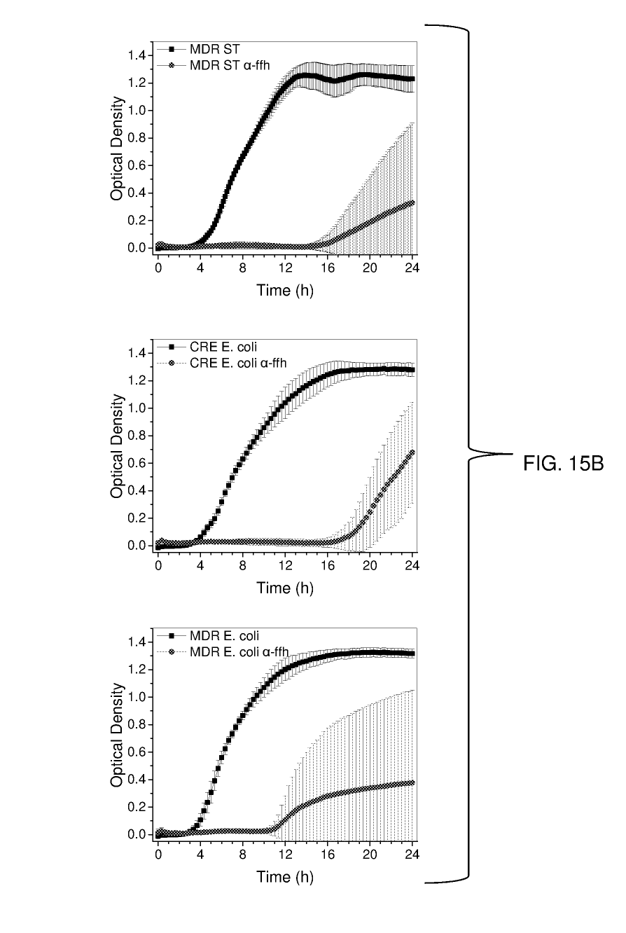

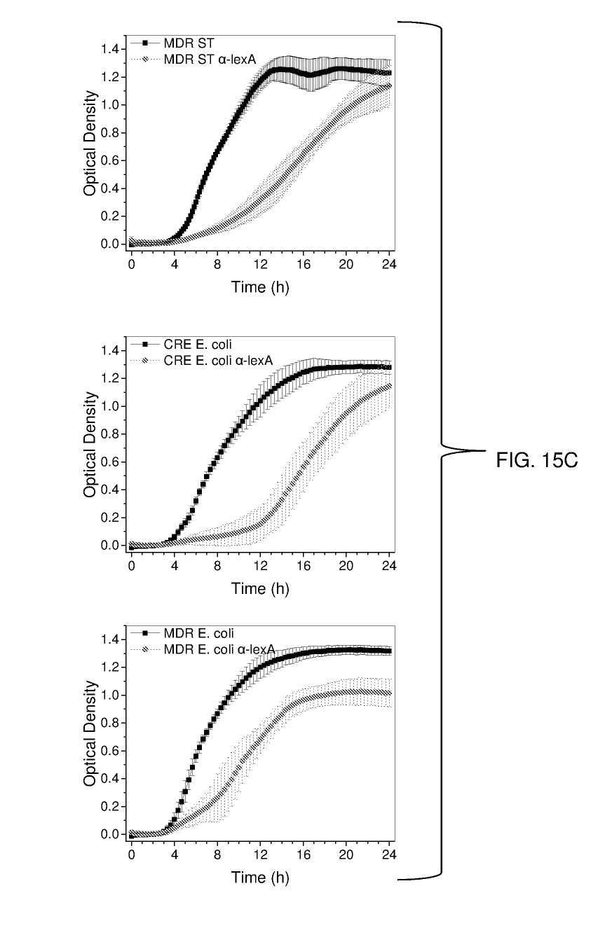

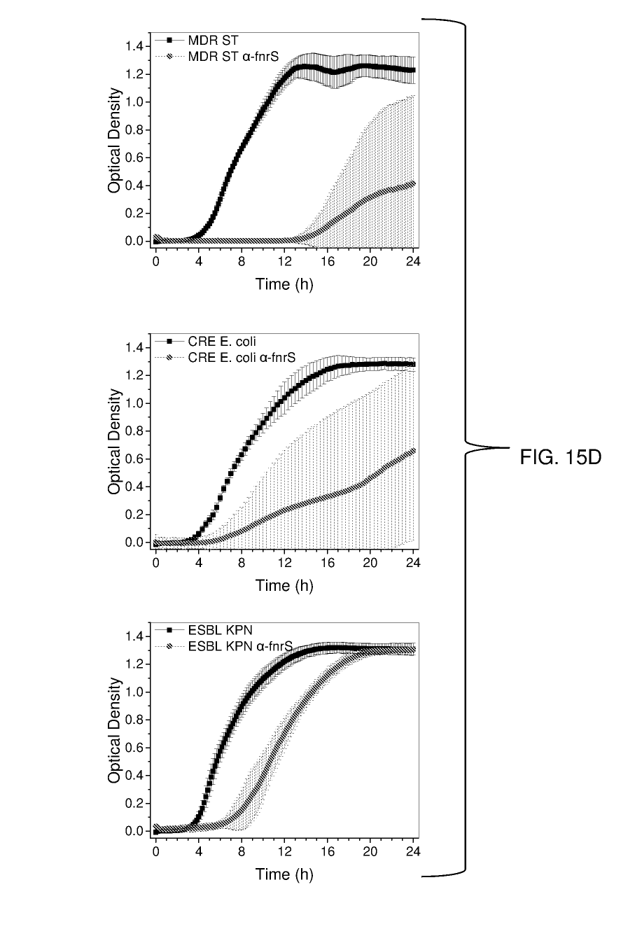

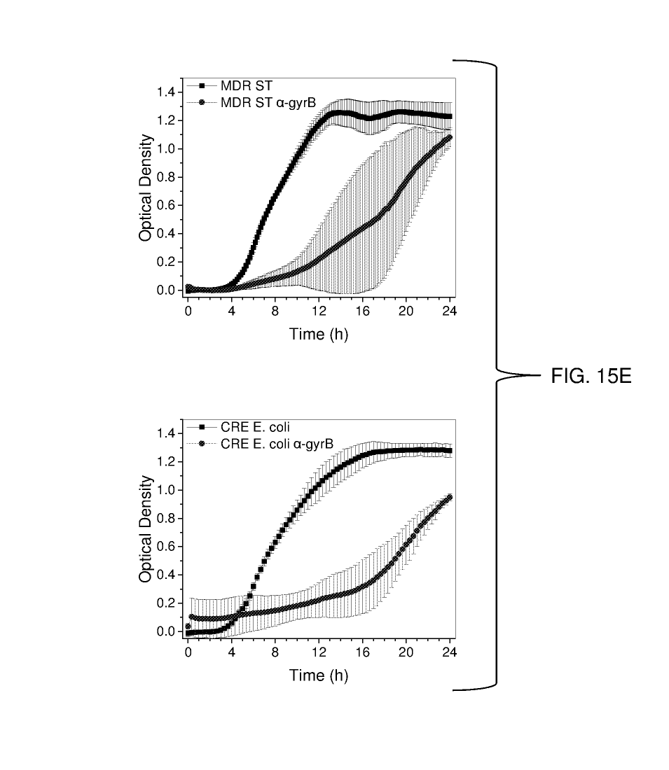

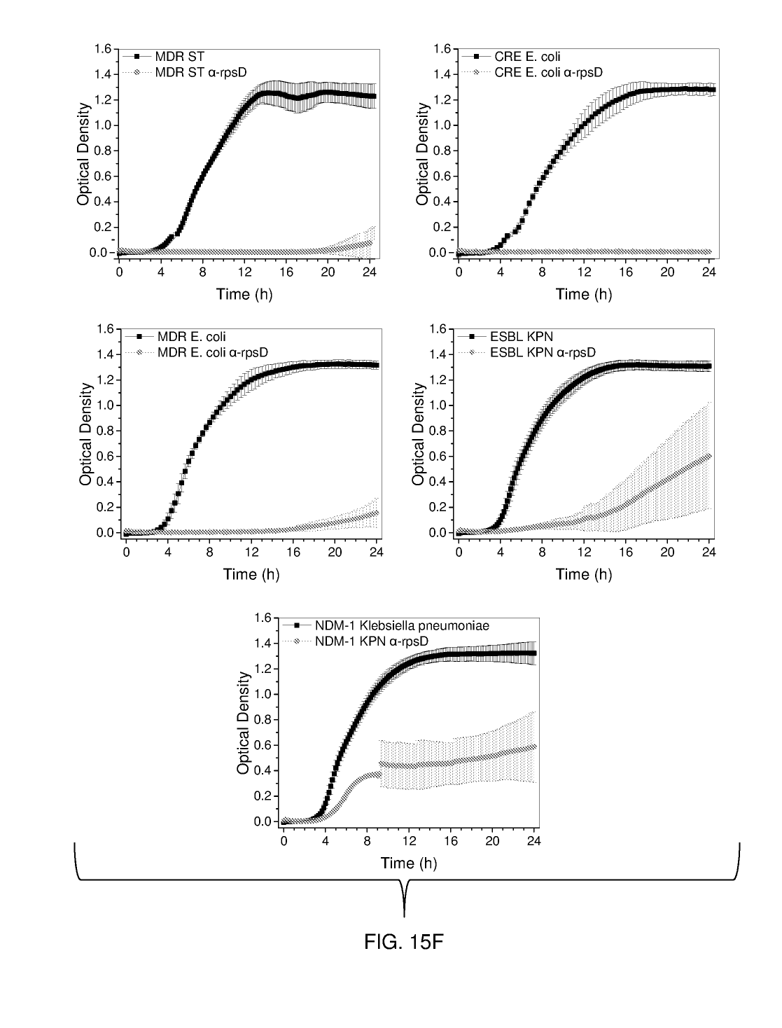

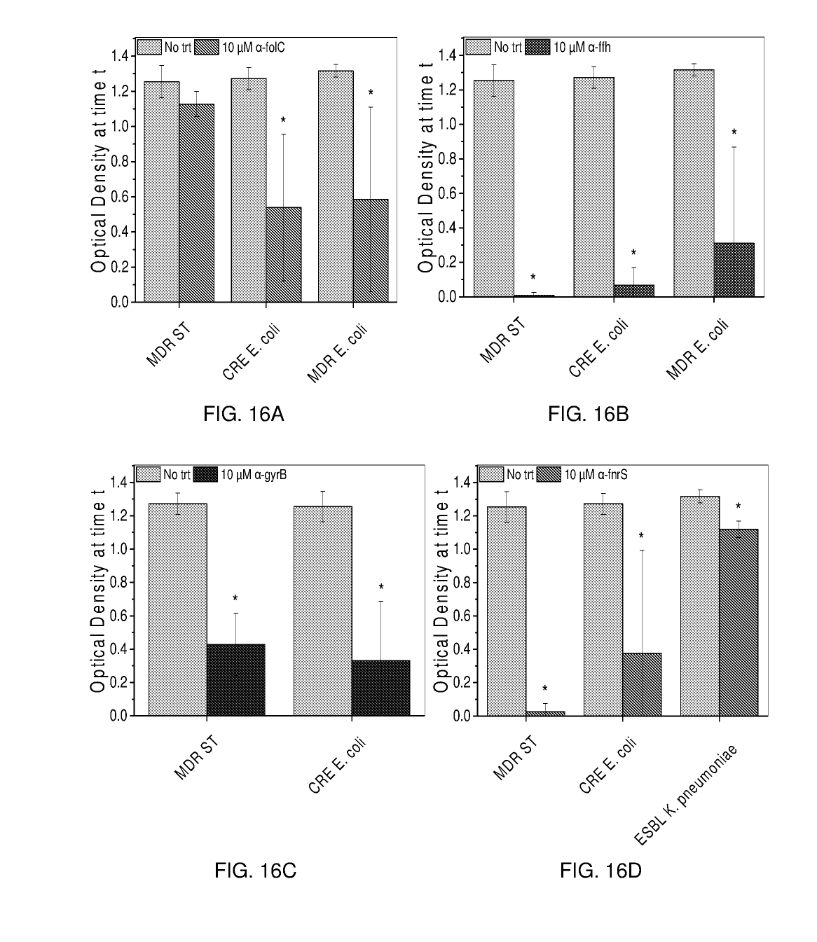

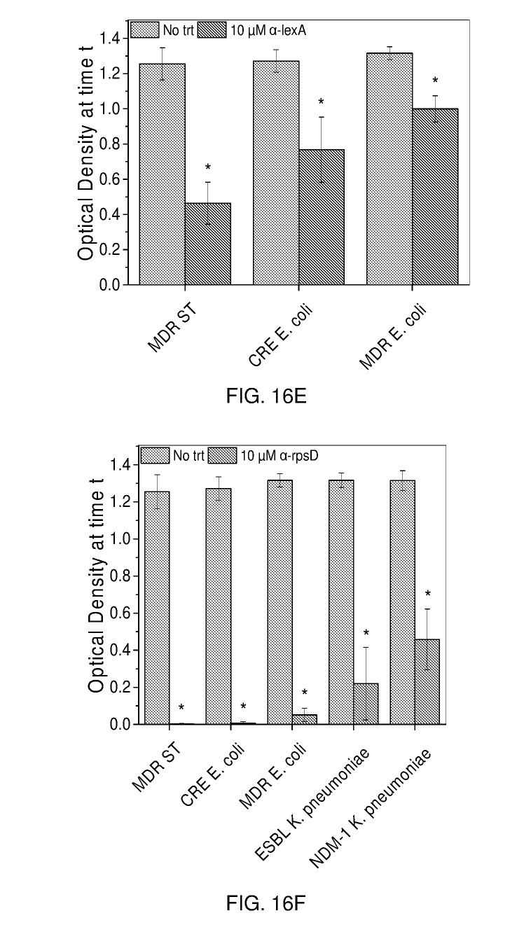

As shown in FIGS. 15-16, MDR S. typhhimurium and CRE E. coli were inhibited by all six antisense oligomers tested (α-folC, α-ffh, α-lexA, α-fnrS, α-gyrB, and α-rpsD). MDR E. coli was inhibited by four of the six oligomers tested (α-folC, α-ffh, α-lexA, and α-rpsD), EBSL K. pneumoniae was inhibited by two of the antisense oligomers tested (α-fnrS and α-rpsD), and NDM-1 K. pneumoniae was inhibited by α-rpsD (FIGS. 15-16). These results show the ability of antisense inhibitors to target novel, non-traditional antibiotic pathways. Because antisense antibiotic are sequence specific and can be designed to not have any mammalian targets, they are non-toxic to mammalian cells, as demonstrated in FIG. 18. This allows for several antisense inhibitors to be administered at the same time, providing broad-range antibiotic activity across a number of common drug resistant bacteria.

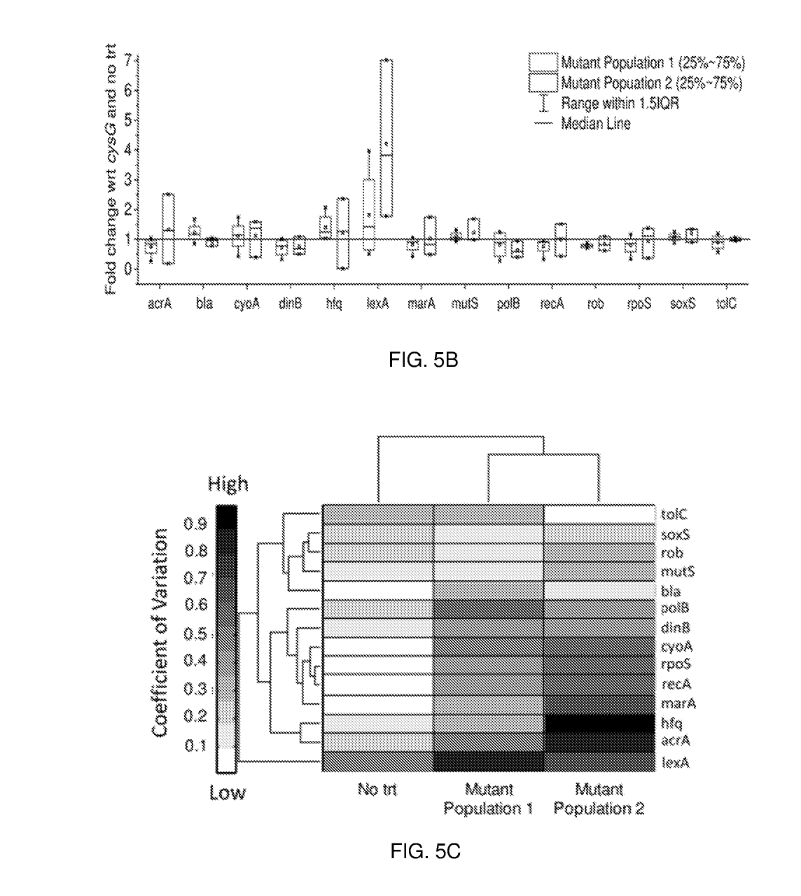

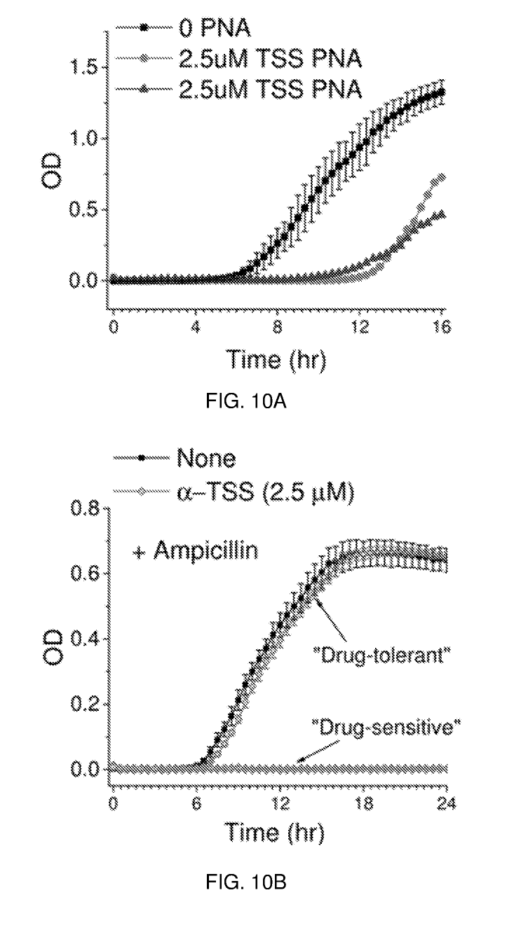

To investigate whether treatment with antisense oligomers would result in adaptive resistance, ampicillin-resistant E. coli cultures were pretreated overnight with bla α-TSS RNA inhibitor and then subjected to selection pressure using 2.5 μM α-TSS and 300 μg/ml ampicillin for 24 hours. In a set of 35 independent cultures, only two cultures developed tolerance to the α-TSS/ampicillin combination after 24 hours (FIG. 10A). This is a rate of adaptive resistance much lower than that commonly seen with ampicillin alone. The two mutant populations were confirmed to be stable mutants having resistance to α-TSS (FIG. 10B). By examining the expression of stress responsive genes in the mutants using qPCR (FIG. 5B), it possible to identify those genes having high variation, indicating involvement in development of adaptive resistance. These genes can then be targeted by antisense oligomers. In certain embodiments, a fold increase in expression of about 2 or more for a particular gene is indicative of that gene being involved in adaptive antibiotic resistance.

None of the independent cultures grown in the presence of 5 μM α-TSS/300 μM ampicillin showed emergence of tolerance to the therapeutic combination. Together, these results show that while adaptive resistance can still emerge with the use of antisense inhibitors, resistant mutants emerge at a lower rate in the presence of antisense inhibitor relative to traditional antibiotics alone, and adaptive resistance can be addressed by targeting those genes identified to be involved in the resistance or by using a sufficiently high concentration of the antisense inhibitor.

As shown and described herein, antisense inhibitors provide an opportunity for mitigating the first sign of emergence of antibiotic resistance with quick development of antisense based inhibitors which require only the sequence of an identified target site and synthesis of the cognate antisense oligomer. Antisense nucleic acid therapeutics have the potential to target any gene in the genome. This allows for combination therapies that target not only multiple therapeutic targets, but also stress response genes which aid in resistance. Targeting resistance head-on rather than waiting for it to develop is useful in designing antibiotics that prevent resistance and remain efficacious for years to come.

Antisense Oligomers

hi particular embodiments described herein, at least one antisense oligomer is designed against a target site on a DNA, RNA, or mRNA sequence associated with antibiotic resistance, where the antisense oligomer is designed to be complementary to the target site. In other embodiments, at least one antisense oligomer is designed against a target site on DNA, RNA, or mRNA sequence associated with an essential target gene involved in a non-traditional antibiotic pathway.

The antisense oligomer can be a nucleic acid, such as RNA, or a nucleic acid analog, including but not limited to peptide nucleic acid (PNA), morpholino, locked nucleic acid (LNA), bridged nucleic acid (BNA), phsophorothioate oligonucleotides, and 2′-O-methyl-substituted RNA. The use of a nucleic acid analog is advantageous in that while they have a different backbone sugar or, in the case of PNA, an amino acid residue in place of the ribose phosphate, they are still capable of binding RNA or DNA according to Watson-Crick pairing, but are immune to nuclease activity.

In certain embodiments, the antisense oligomer is a morpholino. Morpholinos have a backbone of methylenemorpholine rings and phosphorodiamidate linkages, the nucleic acids being bound to the methylenemorpholine rings. Because of their synthetic backbones, morpholinos are not recognized by cellular proteins, and are not degraded by nucleases.

In certain embodiments, the antisense oligomer is an LNA. LNA is a constrained RNA analog having a methylene bridge between the 2′ and 4′ positions in the ribose ring. Due to its constrained backbone, LNA has a high affinity for single-stranded DNA/RNA compared to other analogs. In addition to high affinity, LNAs display high in vivo stability and slower renal clearance, although in rare cases hepatotoxicity has been observed. The increased affinity allows LNA to be used in much shorter oligonucleotide than for many other analogue types.

In certain embodiments, the antisense oligomer is a BNA. BNA monomers can contain a five-, six-, or even a seven-membered bridged structure with a fixed C₃′-endo sugar puckering. The bridge is synthetically incorporated at the 2′, 4′-position of the ribose to afford a 2′, 4′-BNA monomer. An increased conformational inflexibility of the sugar moiety in BNA oligonucleotides results in a gain of high binding affinity with complementary single-stranded RNA and/or double-stranded DNA. BNAs are useful for the detection of short DNA and RNA targets, are capable of single nucleotide discrimination, and are resistant to exo- and endonucleases resulting in high stability for in vivo and in vitro applications.

In certain embodiments, the antisense oligomer is a phosphorothioate (PS) oligonucleotide. In a PS backbone, a sulfur atom replaces one non-bridging oxygen atom and increases nuclease resistance. PS linkage reduces RNA-target affinity somewhat but enhances interaction with plasma proteins, decreasing renal clearance rates. There are also some concerns of possible toxic side effects at higher concentrations.

In certain embodiments, the antisense oligomer is a 2′-O-methly nucleotide. In a 2′-O-methly nucleotide, a methyl group replaces a hydrogen atom in the 2′-hydroxyl group in the ribose ring of RNA, imparting nuclease resistance and inhibiting RNAse-H activation, leaving target RNA intact. Although the 2′-O-methyl modification is insensitive to endonucleases, it is still partially susceptible to exonuclease degradation. By combining PS linkages and 2′-O-methyl nucleotides, much greater in vivo stability has been achieved

In a particular embodiment, the antisense oligomer is a PNA. PNA's backbone is comprised of repeating N-(2-aminoethyl)-glycine units linked by peptide bonds. Purine and pyrimidine bases are linked to the backbone by a methylene bridge (—CH₂—) and a carbonyl group (—(C═O)—). Use of antisense PNA as the antisense oligomer rather than RNA has several advantages. Because the backbone of PNA contains no charged phosphate groups, the binding between PNA/RNA or PNA/DNA strands is stronger than between RNA/RNA or RNA/DNA strands due to the lack of electrostatic repulsion. The neutral PNA backbone also results in the binding being practically independent of the salt concentration. In addition to having increased binding affinity, PNAs are known to bind RNA or DNA with increased specificity.

The genetic DNA sequence, or RNA/mRNA sequences associated with antibiotic resistance can be any sequence that confers antibiotic resistance, or aids in development of resistance. In other embodiments, the RNA sequence associated with antibiotic resistance is a regulatory RNA affecting the antibiotic resistance of bacteria. In particular embodiments, the RNA sequence is a small RNA (sRNA) sequence. Regulatory RNAs can affect antibiotic resistance of bacteria by regulating, for example, RNA synthesis, protein synthesis, cell membrane integrity, membrane transporters, and cell wall turnover. These processes and mechanisms are known to be involved in antibiotic resistance of bacteria.

In other embodiments, the mRNA sequence associated with antibiotic resistance encodes a resistance-inducing enzyme. Resistance-inducing enzymes that can be targeted by antisense oligomers, thereby re-sensitizing bacteria to the original antibiotic, include but are not limited to β-lactamases, NDM-1, carbapenemase, aminoglycoside acetyltransferase, and dihydropteroate synthase. In other embodiments, the mRNA sequence associated with antibiotic resistance can include, but is not limited to mecA, pbp1, pbp2, pbp3, orf-X-mecI, mecI-ccrA1, arsC, CraA, rpoB, katG, inhA, amvA, and adeQYZ.

In other embodiments, a genetic DNA sequence to be targeted disrupts transcription of an essential gene associated with a non-traditional antibiotic pathway. These same pathways can also be disrupted by targeting the essential gene's mRNA. Non-traditional antibiotic pathways include but are not limited to transport and membrane function or biosynthesis, metabolism, redox homeostasis, stress response, cell signaling, replication and growth, transcription and translation, and DNA modifications, repair, and maintenance.

Essential genes in transport and membrane function or biosynthesis include cdsA, encoding CDP-diglyceride synthetase, which is a common intermediate in the biosynthesis of phospholipids. Essential gene msbA encodes a protein in the ATP binding cassette (ABC) superfamily of transporters and functions to transport lipids between the inner and outer membrane. The gene lptA is essential due to its role in the ABC superfamily and as a lipopolysaccharide transporter. Gene sgrT is essential and functions as an inhibitor of glucose uptake. Folate balance in maintained in E. coli by the essential gene folC. The gene family of secA, secD, secE, secF, secM, and secY are all essential due to their role in the Sec protein secretion complex.

Within metabolism there are many examples of essential genes. The essential gene adk encodes adenylate kinase which functions in energy homeostasis and the interconversion of adenine nucleotides. Gene coaD is essential in metabolism for its encoded product which is a key component in the synthesis of coenzyme A, an enzyme in the citric acid cycle. The gene eno is essential due to its role as a component in enolase, involved in glycolysis, and in RNA degradosomes. The gene family of ispA, ispB, ispD, ispE, ispF, ispG, ispH, and ispU is essential for their role in isoprenoid biosynthesis.

Within redox homeostasis, stress response, and cell signaling there are few essential genes in E. coli. The gene can functions in redox homeostasis and is a component of carbonic anhydrase 2 which functions in carbon dioxide and acid balance. Within stress response, grpE is an essential gene that functions as a heat shock protein. The transcriptional repressor of the SOS regulon, lexA, is also an essential gene in stress response. Other targetable stress response genes include, but are not limited to, marA, acrA, tolC, rpoS, cyoA, hfq, dinB, polB, mutS, lexA, rob, soxS, and recA. The gene rseP, encoding zinc metallopeptidase, activates rpoE by degrading its repressor. Essential components of the signal recognition particle are encoded by the essential genes ffh and ffs. The genes lepB and lspA are essential for their role as signal peptidase I and II, respectively, which modifying secretory and membrane proteins.

There are many essential genes in E. coli involved in replication and growth. Two essential genes which code for GTP-binding proteins are era and odgE which are essential for cell growth and DNA replication, respectively. The essential gene family of ftsA, ftsB, ftsE, ftsI, ftsK, ftsL, ftsQ, ftsW, ftsY, and ftsZ function as cell division proteins. The holA and holB genes are essential due to their function as DNA polymerase III subunits.

For the central dogma processes inside cells, transcription and translation, there are many essential genes. Essential genes bamA and bamD are components of the beta-barrel assembly machine which are part of the outer membrane assembly complex. The genes gyrA and gyrB are essential due to their role as subunit A and B, respectively, of DNA gyrase. The peptide chain release factor coded by prfA is essential as a translation termination factor in E. coli. The essential gene family comprised of rpsA, rpsB, rpsC, rpsD, rpsE, rpsH, rpsJ, rpsK, rpsL, rpsN, rpsP, rpsR, and rpsS are protein subunits that comprise the 30S ribosomal subunit complex.

Within DNA modifications, repair, and maintenance there are few essential genes. DNA ligase is coded for by the essential gene ligA which plays a role in repair DNA breaks. Another essential gene is prmC which encodes protein-(glutamine-N5) methyltransferase and methylate's class 1 translation termination release factors on glutamine residues. The gene trmD is essential for its role as a component of tRNA (guanine-1-)-methyltransferase.

In other embodiments, other essential genes can also be targeted. The gene fnrS encodes an essential non-coding hfq-binding small RNA which is upregulated under anaerobic conditions. An essential gene ilvX encodes a small protein with unknown function that is detected during stationary phase. The essential gene apbE encodes a predicted lipoprotein which is required for thiamine biosynthesis. Other targetable essential genes include, but are not limited to, nusA, rpoD, nusE, ffh, rpsU, accD, degS, ftsN, lolA, hflB, mraY, rsG, rplV, nadD, murF, murA, and mreD.

It will be recognized by those of skill in the art that any of the DNA or mRNA sequences described above can be targeted by antisense inhibitors. Target sequences can be those of E. coli or the homologous gene or mRNA sequence in another target bacterium. Given the benefit of this disclosure, those of skill in the art will be able to identify a target sequence and design an antisense inhibitor oligomer to target the gene or mRNA sequence.

Target sites on DNA or RNA (e.g. sRNA) associated with antibiotic resistance can be any site to which binding of an antisense oligomer will inhibit the function of the DNA or RNA sequence. Inhibition can be caused by steric interference resulting from an antisense oligomer binding the DNA RNA sequence, thereby preventing proper transcription of the DNA sequence or translation of the RNA sequence.

Target sites on an essential gene associated with an antibiotic pathway can be any site to which binding of an antisense oligomer will inhibit transcription of the gene. In certain embodiments, antisense sequences are designed to be centered around the start codon of a target gene.

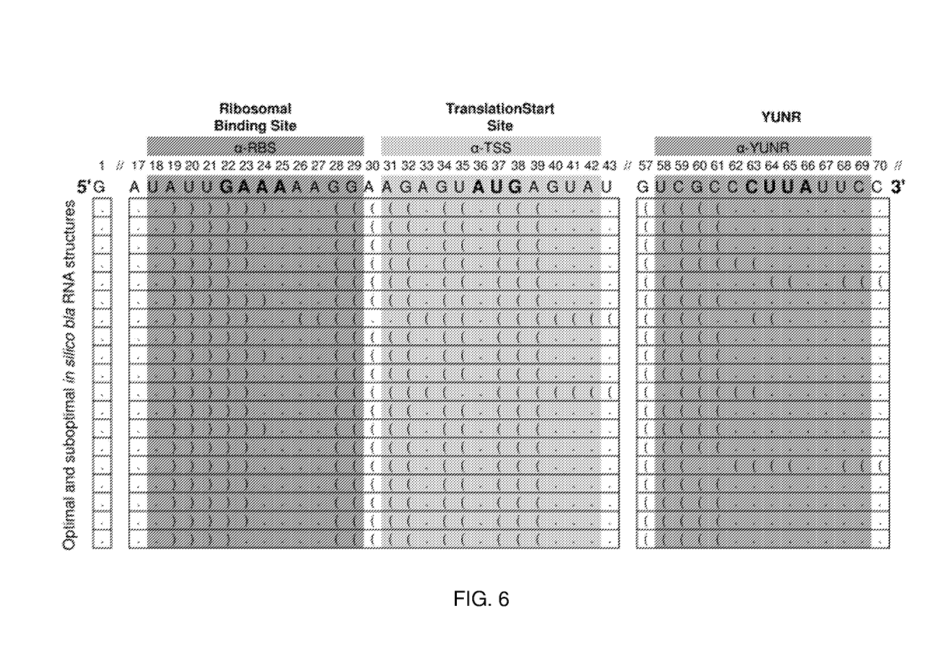

Target sites on an mRNA sequence associated with antibiotic resistance can be any site to which binding of an antisense oligomer will inhibit expression of the mRNA sequence. Protein functional sites on mRNA have been shown to be effective antisense sites for blocking ribosomal binding and migration (27). These include the ribosomal binding site (RBS) and translation start site (TSS), which are located in the 5′ untranslated region (UTR). In particular embodiments, the target site is a ribosomal binding site (RBS), a translational start site (TSS), or a YUNR motif. Targeting an RBS inhibits ribosomal binding to the mRNA, thereby preventing translation of the mRNA, while targeting a TSS prevents a bound ribosome from migrating past the start codon, thereby inhibiting translation. Targeting a 5′ YUNR motif results in a rate-limiting interaction between mRNA and ribosome, and can prevent ribosomal migration, thereby inhibiting mRNA translation. FIG. 6 depicts optimal and suboptimal RNA structures of the bla mRNA target regions.

An antisense oligomer can be complementary to a single target site or two or more target sites. For example, an antisense oligomer can be complementary to any one of a TSS, RBS, or YUNR motif. However, an antisense oligomer can be complementary to two or more of the target sites. In particular embodiments, each individual antisense oligomer is complementary to a single target site. Wherein each individual antisense oligomer is complementary to a single target site, the antisense oligomer can be about 10-mers to about 20-mers in length. In certain embodiments, the antisense oligomer is about 12-mers in length. In certain embodiments, the antisense inhibitory oligomers are designed with the target sequence in the middle of the oligomer, with 3-5 nucleotides flanking the overlapping region. This provides for antisense oligomers with both high affinity and specificity. Wherein an individual antisense oligomer is complementary to two or more target sites, the antisense oligomer can be up to about 40-mers in length.

Because of the neutral PNA backbone, antisense PNAs tend to be hydrophobic. This hydrophobicity can impede uptake of the antisense PNA into bacterial cells. In order to overcome this, an antisense PNA can be linked to a cell penetrating peptide. Many cell penetrating peptide are known in the art, including but not limited to (KFF)₃K (SEQ ID NO: 49), penetratin, NLS, TAT, Arg(9), D-Arg(9), 10HC, cyLoP-1, Pep-1, and those cell penetrating peptides describe in U.S. Pat. No. 9,238,042 (Frederick et al., 2016), and can be linked to an antisense antibiotic oligomer. In particular embodiments, the cell penetrating peptide is (KFF)₃K (SEQ ID NO: 49).

In particular embodiments, the antisense inhibitory oligomer can comprise a linker, such as an O-linker, to reduced steric interference in target binding. Other linkers known in the art, including but not limited an E-linker, a C6A linker, a C6SH linker, an X-linker, and a C11SH linker, can also be used. In a particular embodiment, an antisense PNA comprises a cell penetrating peptide linked to the antisense PNA sequence via an O-linker, as shown in Table 1.

In certain embodiments, antisense oligomers are designed against target sites proximal to the 5′ UTR of β-lactamase enzyme-encoding bla mRNA. As described herein, these include bla α-RBS (C-ATAACTTTTTCC-N(SEQ ID NO: 1); RBS in bold), bla α-TSS (C-TCTCATACTCAT-N (SEQ ID NO: 3); translational start site in bold), and bla α-YUNR (C-AGCGGGAATAAG-N (SEQ ID NO: 5); YUNR in bold) (see Examples). In certain embodiments, bla α-RBS, bla α-TSS, and bla α-YUNR comprise a cell penetrating peptide linked to the antisense inhibitor oligomer via an O-linker (Table 1).

TABLE 1
Antisense antibiotic sequences and oligomers targeting bla mRNA. The sequences are
written from the N-terminus to the C-terminus.

| Target | | | SEQ | | SEQ | |
|---|---|---|---|---|---|---|
| gene | | Antisense | ID | | ID | |
| mRNA | Molecule | Sequence | NO: | Antisense Antibiotic Oligomer | NO: | mRNA Target Site |
| bla | α-RBS | cctttttcaata | 1 | KFFKFFKFFK-O-cctttttcaata | 2 | Ribosomal binding site |
| mRNA | | | | | | |
| bla | α-TSS | tactcatactct | 3 | KFFKFFKFFK-O-tactcatactct | 4 | Translation start site |
| mRNA | | | | | | |
| bla | α-YUNR | gaataagggcga | 5 | KFFKFFKFFK-O-gaataagggcga | 6 | YUNR sequence motif |
| mRNA | | | | | | |

Antisense sequences are all twelve peptide nucleic acids long, centered around the mRNA target site. Antisense antibiotic oligomers comprise the antisense sequence linked to a cell penetrating peptide such as (KFF)₃K (SEQ ID NO: 49) via an O-linker. Antisense antibiotic oligomers can comprise RNA or a nucleic acid analog such as PNA, morpholino, LNA, BNA, GNA, TNA, or 2′-O-methyl-substituted RNA

In other embodiments, antisense inhibitor oligomers are designed against a target gene selected from the group of folC, ffh, lexA, fnrS, rpsD, and gyrB. In certain embodiments, the antisense oligomers comprise a cell penetrating peptide linked to the antisense inhibitor oligomer via an O-linker. Sequences for such antisense inhibitor oligomers are provided in Table 2.

TABLE 2
Antisense antibiotic sequences and oligomers designed to target novel antibiotic targets.
The sequences are written from the N-terminus to the C-terminus.

| | | SEQ | | SEQ | | | |
|---|---|---|---|---|---|---|---|
| Gene | Antisense | ID | | ID | Targeted | # off | |
| Target | Sequence | NO: | Antisense Antibiotic Oligomer | NO: | Pathway | targets | Homology |
| folC | taatcatggtat | 7 | KFFKFFKFFK -O-taatcatggtat | 8 | Metabolism | 0 | E. coli |
| | | | | | | | K. pneumoniae |
| Ffh | tcaaacattgtc | 9 | KFFKFFKFFK -O-tcaaacattgtc | 10 | Signal recognition | 0 | E. coli |
| | | | | | particle | | K. pneumoniae |
| | | | | | | | S. enterica |
| lexA | gctttcattccg | 11 | KFFKFFKFFK -O-gctttcattccg | 12 | Regulator of stress | 0 | E. coli |
| | | | | | response | | K. pneumoniae |
| | | | | | | | S. enterica |
| fnrS | cacctgcaagag | 13 | KFFKFFKFFK-O-cacctgcaagag | 14 | Small Hfq binding | 1 | E. coli |
| | | | | | RNA | | |
| gyrB | ttcgacatcaac | 15 | KFFKFFKFFK-O-ttcgacatcaac | 16 | Protein | 1 | E. coli |
| | | | | | biosynthesis | | K. pneumoniae |
| | | | | | | | S. enterica |
| rpsD | ttgccattttct | 17 | KFFKFFKFFK-O-ttgccattttct | 18 | DNA replication | 3 | E. coli |
| | | | | | | | K. pneumoniae |
| | | | | | | | S. enterica |

Antisense sequences are all twelve peptide nucleic acids long, centered around the start codon of the target gene. Antisense antibiotic oligomers comprise the antisense sequence linked to a cell penetrating peptide such as (KFF)₃K via an O- linker. Antisense antibiotic oligomers can comprise RNA or a nucleic acid analog such as PNA, morpholino, LNA, BNA, GNA, TNA, or 2′-O-methyl-substituted RNA.

It will be recognized that antisense oligomers can be similarly designed to target genes and mRNA encoding other enzymes and proteins associated with drug-resistance, as well as RNA sequences (e.g., regulatory sRNA) associated with drug-resistance. While some of these other enzymes and proteins and their associated genes are described above, with the benefit of the present disclosure, those of skill in the art will be able to employ the antisense inhibitor strategy described herein to target other non-traditional antibiotic pathways.

The antisense antibiotic oligomers used in accordance with this invention can be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of one of ordinary skill in the art.

Compositions

Certain embodiments described herein provide a composition for re-sensitizing bacteria to one or more antibiotics. Other embodiments describe a composition having bactericidal or bacteriostatic effects. Yet other compositions described herein prevent the emergence of antibiotic resistance. The compositions comprise at least one antisense inhibitor oligomer described herein.

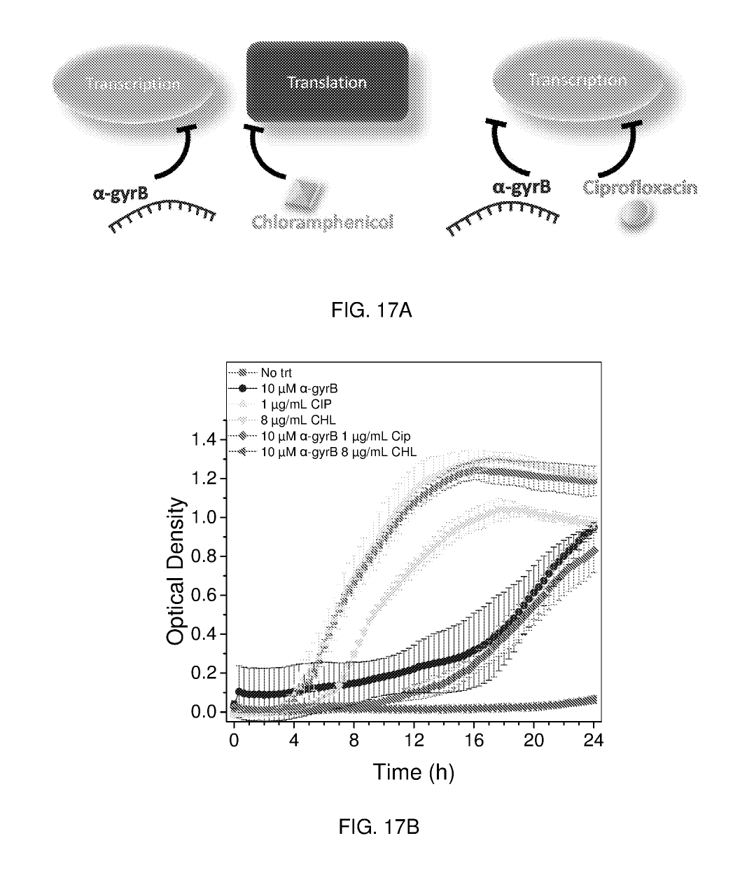

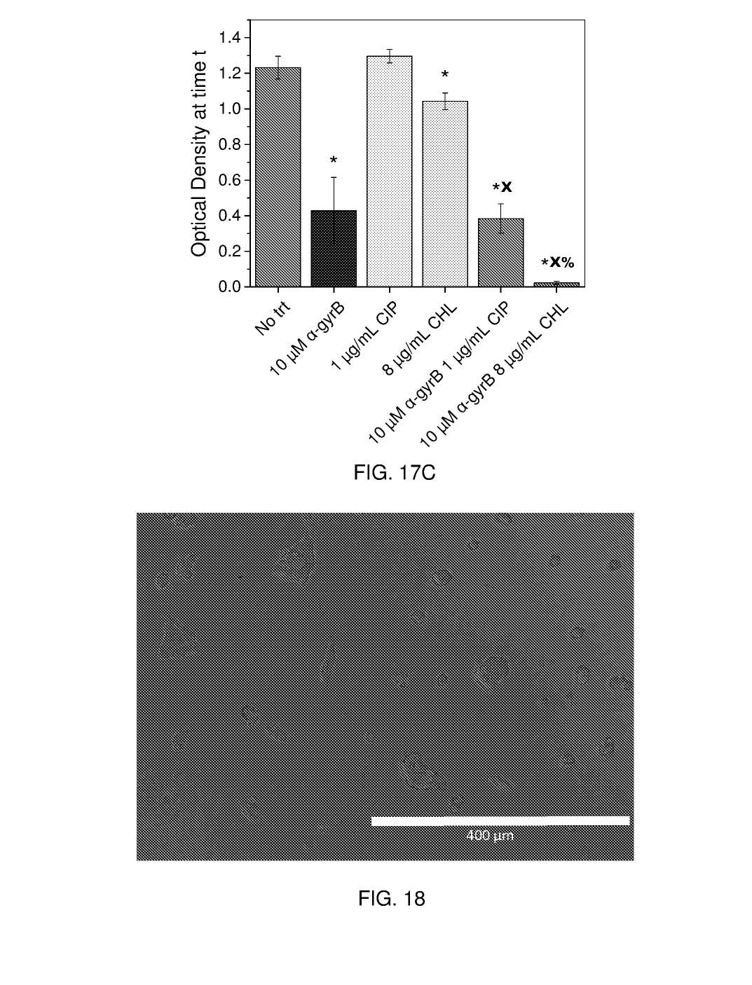

A composition of the present disclosure can comprise one or more antisense oligomers. Wherein the composition comprises a single antisense oligomer, the antisense oligomer is capable of re-sensitizing bacteria to an antibiotic, has bactericidal effects, or has bacteriostatic effects. For example, a composition can comprise bla α-TSS, which binds to the translational start site of bla mRNA. By preventing translation of bla mRNA into β-lactamase, bla α-TSS effectively re-sensitizes β-lactam resistant bacteria to β-lactam antibiotics. Alternately, the antisense oligomer has a bactericidal effect on its own. A-rpsD, for example, is capable of reducing the number of MG1655 E. coli colony forming units per milliliter to zero after only two hours of treatment. In other embodiments, the antisense inhibitor oligomer has bacteriostatic effects, such as α-lexA, which prevents significant increases in viable MG1655 E. coli cells at six and eight hours.

Wherein the composition comprises two or more antisense oligomers, the antisense oligomers can be designed to target different targets in different pathways, different targets in the same pathway, or the same target in the same pathway (see FIG. 17A). As shown in FIG. 17A, α-gyrB can be used alongside chloramphenicol, where the antisense inhibitor α-gyrB prevents transcription of DNA gyrase, while the small molecule antibiotic chloramphenicol targets the 23s rRNA of the 50s ribosomal subunit, preventing translation. In certain embodiments, the different targets can be different target sites on the same mRNA sequence. For example, a composition can comprise antisense oligomers that are complementary to an RBS, TSS, or YUNR motif, or a combination thereof on the same mRNA sequence. In other embodiments the different targets can be on unique mRNA sequences. In these embodiments, it is possible to target different enzymes or proteins associated with drug resistance. For example, the two or more antisense oligomers target an RBS, TSS or YUNR motif of an mRNA of at least one drug resistance-inducing enzyme, as well the mRNA of at least one stress response gene or other non-traditional antibiotic pathway-associated gene associated with drug resistance. In yet another embodiment, a composition comprising two or more antisense oligomers comprises at least two antisense oligomers that target different target sites on the same mRNA, such as an RBS, TSS, YUNR motif, or combination thereof, and at least one oligomer that targets a different mRNA.

Wherein the antisense oligomer's target is an RNA sequence (e.g., sRNA), it is possible to target different segments of the same RNA sequence. In particular embodiments, the target segments of the RNA overlap.

Also provided herein are compositions comprising 10 or more unique antisense oligomers. In such an embodiment, one or more antisense oligomers target at least one target site on a first RNA or mRNA, while the remaining antisense oligomers target unique RNAs or mRNAs, or different target sites on the first RNA or MRNA. In the case of mRNA, such compositions employ the strategy of preventing translation of several proteins associated with drug resistance, thereby ensuring, for example, re-sensitization to an antibiotic. Such compositions can also have bactericidal or bacteriostatic effects, or can prevent emergence of antibiotic resistance. In certain embodiments wherein the composition comprises 10 or more unique antisense oligomers, all antisense oligomers target the same mRNA or RNA sequence. The 10 or more antisense oligomers can target one or more target sites on the mRNA or RNA sequence.

The at least one antisense oligomer is present in the composition at a pharmaceutically effective concentration. The pharmaceutically effective concentration of an antisense oligomer will depend on several factors, including but not limited to the oligomer's backbone composition, the affinity of the oligomer for its target, the specificity of the oligomer for its target, and the ability of the oligomer to enter the cell. In certain embodiments, a pharmaceutically effective concentration of an antisense oligomer is that concentration that prevents development of adaptive resistance. In particular embodiments, wherein the antisense oligomer is an antisense PNA, its pharmaceutically effective concentration can be from 0.5 μM to 40 μM. Wherein the antisense oligomer is bla α-TSS, the pharmaceutically effective concentration is about 1.5 μM-5 μM. Wherein the antisense oligomer is bla α-RBS, the pharmaceutically effective concentration is between about 5 μM-25 μM. Wherein the antisense oligomer is α-folC, α-ffh, α-lexA, α-fnrS, α-gyrB, α-rpsD, the pharmaceutically effective concentration is between about 5 μM-15 μM.

Compositions provided herein can further comprise at least one conventional antibiotic. In certain embodiments, the antibiotic included in the composition is one which the at least one antisense oligomer is designed to re-sensitize bacteria to. For example, a composition can comprise one or more antisense oligomers selected from the group of bla α-RBS, bla α-TSS, and bla α-YUNR, along with ampicillin. As disclosed herein, bla α-RBS bla α-TSS inhibit translation of bla mRNA, which encodes β-lactamase. By providing at least one of these antisense oligonucleotides along with the β-lactam antibiotic ampicillin, it is possible to re-sensitize β-lactam resistant bacteria to ampicillin and treat the bacterial infection.

In other embodiments, the conventional antibiotic is one that targets the same target of the same pathway. For example, a composition can comprise α-gyrB and ciprofloxacin, both of which target transcription of DNA gyrase (FIG. 17).

In other embodiments, the antibiotic is one that targets a different target and of a different antibacterial pathway. For example, a composition can comprise α-gyrB to inhibit transcription of gyrB and the conventional antibiotic chloramphenicol, which targets the 23s rRNA of the 50s ribosomal subunit (FIG. 17).

As described herein, antisense oligomers can be designed to effectively inhibit transcription of essential genes associated with antibiotic pathways, or inhibit translation of mRNA encoding enzymes and proteins associated with drug resistance. However, antisense oligomers may not always completely inhibit such transcription or translation. Wherein antisense oligomers result in partial or incomplete inhibition of translation, at least one antibiotic-associated pharmaceutical inhibitor can be included in the composition. For example, wherein a composition comprises one or more antisense oligomers targeted toward mRNA encoding a β-lactamase, it can be beneficial to include in the composition a β-lactamase inhibitor, such as clavulanic acid, sulbactam, or tazobactam. Certain compositions can include both an antibiotic and an associated pharmaceutical inhibitor.

As described above, the antisense antibiotic oligomers can be used in a synergistic combination with other known antimicrobial agents, including but not limited to penicillins, cephalosporins, carbacephems, cephamycins, carbapenems, monobactams, aminoglycosides, glycopeptides, quinolones, tetracyclines, macrolides, and fluoroquinolones. Examples of antibiotic agents include, but are not limited to, Penicillin G (CAS Registry No.: 61-33-6); Methicillin (CAS Registry No.: 61-32-5); Nafcillin (CAS Registry No.: 147-52-4); Oxacillin (CAS Registry No.: 66-79-5); Cloxacillin (CAS Registry No.: 61-72-3); Dicloxacillin (CAS Registry No.; 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefinetazole (CAS Registry No.: 56796-20-4); Cefotetan (CAS Registry No.: 69712-56-7); Cefprozil (CAS Registry No.: 92665-29-7); Lincomycin (CAS Registry No.: 154-21-2); Linezolid (CAS Registry No.: 165800-03-3); Loracarbef (CAS Registry No.: 121961-22-6); Cefetamet (CAS Registry No.: 65052-63-3); Cefoperazone (CAS Registry No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazidime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7); Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; and their various salts, acids, bases, and other derivatives.

In certain embodiments, the composition is a pharmaceutical composition. The compositions may routinely contain pharmaceutically acceptable concentrations of salt, buffering agents, preservatives and various compatible carriers or diluents. The proportion and identity of the pharmaceutically acceptable diluent is determined by chosen route of administration, compatibility with the antisense oligomers of the composition, and standard pharmaceutical practice. Generally, the pharmaceutical composition will be formulated with components that will not significantly impair the biological activities of the antisense oligomers.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the compositions are prepared by uniformly and intimately bringing into association the active ingredients with liquid carriers or finely divided solid carriers or both, and then, if necessary, shaping the product. One of skill in the art will recognize that formulations are routinely designed according to their intended use, i.e. route of administration.

Methods to Re-Sensitize Bacteria to an Antibiotic and to Treat a Bacterial Infection

A subject can be re-sensitized to an antibiotic utilizing an appropriate composition disclosed and described herein. Wherein the re-sensitizing composition further comprises an antibiotic, the subject can be treated of bacterial infection. Wherein the composition comprises one or more antisense oligomers having bactericidal or bacteriostatic effects, a subject can be treated of bacterial infection by administering an appropriate dose of the composition. Optionally, such compositions can comprise one or more conventional antibiotics. Compositions described herein can be administered similarly to currently available antibiotics, including but not limited to oral administration, nasal administration, intravenous administration, intramuscular administration, intraperitoneal administration, topical administration, local delivery methods, and in feed and water supplies.

Subjects to be re-sensitized to an antibiotic or to be treated for a bacterial infection can be selected from the group of: human; feed animals including but not limited to cattle, swine, poultry, goat, and sheep; companion animals including but not limited to dog, cat, rodent, bird, and reptile; and laboratory animals. Subjects to be re-sensitized to an antibiotic can be those who have shown resistance to an antibiotic, or to whom an antibiotic is to be given where there is common drug resistance to the antibiotic. A composition described herein can also be provided to a subject in order to prevent or delay development of antibiotic resistance.

Methods are provided for treating a bacterial infection in a subject in need thereof. Subjects to be treated for a bacterial infection are administered a composition described herein, thereby treating the bacterial infection. In certain embodiments, the composition used for treating a bacterial infection targets at least one mRNA sequence that encodes a protein essential for bacterial homeostasis. In certain embodiments, the composition does not comprise a conventional antibiotic, while in other embodiments, the composition does comprise at least one conventional antibiotic. In yet other embodiments, a composition comprising at least one antisense oligomer capable of affecting translation of at least one drug resistance-associated enzyme or protein is administered to the subject first, followed by administration of an antibiotic. In preferred embodiments, the subject is human.

Also provided herein are methods for preventing emergence of antibiotic resistance in bacteria in a subject in need thereof. Subjects are administered a composition described herein, thereby preventing emergence of antibiotic resistance in bacteria. Preferably, the composition used for treating a bacterial infection targets at least one DNA or mRNA sequence essential for development of antibiotic resistance.

In methods for re-sensitizing bacteria to an antibiotic and for treating a bacterial infection, a pharmaceutically effective amount of the composition is administered for a sufficient time period to achieve a desired result. For example, a composition can be administered in quantities and dosages necessary to deliver at least one antisense oligomer capable of inhibiting translation of mRNA encoding at least one enzyme or other protein associated with antibiotic resistance.

The formulation of therapeutic compositions and their subsequent administration (dosing) is believed to be within the skill of those in the art. Optimal dosing schedules can be calculated from measurements of drug accumulation in the body of a subject. Persons of ordinary skill can easily determine optimum dosages, dosing methodologies and repetition rates. Optimum dosages may vary depending on the relative potency of individual antisense antibiotic oligomers, and can generally be estimated based on EC₅₀s found to be effective in in vitro and in vivo animal models. In general, dosage is from 0.01 μg to 100 g per kg of body weight, and may be given once or more daily, weekly, or monthly.

Persons of ordinary skill in the art can easily estimate repetition rates for dosing based on measured residence times and concentrations of the antisense antibiotic oligomers in bodily fluids or tissues. Following successful treatment, it may be desirable to have the subject undergo maintenance therapy to prevent the recurrence of the disease state, wherein the antisense antibiotic oligomer is administered in maintenance doses, ranging from 0.01 μg to 100 g per kg of body weight.

In certain embodiments, a patient is treated with a dosage of antisense antibiotic oligomer that is at least about 1, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100 mg/kg body weight.

A pharmaceutically effective amount will depend on several factors, including but not limited to the antisense oligomer's backbone composition, the affinity of the antisense oligomer for its target, the specificity of the antisense oligomer for its target, and the ability of the antisense oligomer to enter the cell. Factors can include, among others, the mode of administration, the age, health, and weight of the subject, the nature and extent of the infection, and the frequency of the treatment. One of skill in the art can determine the appropriate effective amount based on the above factors.

Methods for Identifying Target Genes and Developing Antibacterial Antisense Therapeutics

Provided herein are methods for identifying target genes involved in adaptive antibiotic resistance in bacteria. In one embodiment, the method comprises pretreating antibiotic-resistant bacteria with a composition described herein wherein the composition is free of antibiotic and antibiotic-associated pharmaceutical inhibitor, incubating the pretreated antibiotic-resistant bacteria with the composition and an antibiotic to which the bacteria is resistant, selecting one or more colonies of bacteria that exhibit growth, determining the expression of two or more stress response genes, determining a fold increase for each of the two or more stress response genes relative to a control wherein antibiotic-resistant bacteria were not pretreated with the composition, and identifying a target gene involved in adaptive antibiotic resistance, wherein a fold increase of about 2 for a particular gene is indicative of a target gene involved in adaptive antibiotic resistance (see also Examples I and V). In other embodiments, the fold increase is determined as a coefficient of variation of cycle numbers (C_{q}) of the stress response genes measured during qPCR with respect to a housekeeping gene (see Example V). A higher relative COV is indicative of a target gene involved in adaptive antibiotic resistance. In particular embodiments, the stress response genes analyzed are selected from marA, acrA, tolC, rpoS, cyoA, hfq, dinB, polB, mutS, lexA, rob, soxS, and recA.

A gene expression signature of resistance to antisense oligomers described herein provides for the identification of potential targets for the development of other antisense oligomers. Those genes displaying an increase in expression compared to the control can be targeted by designing antisense oligomers that are complementary to mRNA of those genes. This provides a method for continuing to develop therapeutics having the potential to extend the effective life of an antibiotic.

The invention described herein can be practiced, unless otherwise indicated, using conventional methods of chemistry, molecular biology, microbiology, cell biology, and cell culture, which are all within the skill of the art.

### EXAMPLES

The materials, methods, and embodiments described herein are further defined in the following Examples. Certain embodiments of the present invention are defined in the Examples herein. It should be understood that these Examples, while indicating certain embodiments of the invention, are given by way of illustration only. From the discussion herein and these Examples, one skilled in the art can ascertain the essential characteristics of this invention and without departing from the spirit and scope thereof, can make various changes and modifications of the invention to adapt it to various usages and conditions.

### Example I. Materials and Methods

Bacterial Strains and Cell Culture Conditions.

pAKgfp1 plasmid was obtained from Addgene for TEM-1 β-lactamase resistance gene, bla (43). The plasmid was cloned into chemically competent Zymo DH5a E. coli (Expressys). Untransformed Zymo DH5a was used as a control strain for β-lactamase activity assays. Liquid cultures were grown in 2% LB, incubated at 37° C. and shaken at 225 rpm. Solid cultures were grown on 2% LB broth, 1.5% agar at 37° C. Ampicillin sodium salt (Sigma Aldrich) was used for selection at a concentration of 300 μg/mL for MIC and CFU analysis. Optical density measurements were taken using a Tecan GENios at 562 nm with a bandwidth of 35 nm. All bacterial freezer stocks were stored in 40% glycerol at −80° C.

MG1655 E. coli cultures were started from individual colonies from 2% LB and 1.5% agar into M9 media. They were diluted 1:10,000 from overnight culture into fresh M9 with respective treatment and grown at 37° C. with 225 rpm shaking. Clinical strains were started from individual colonies grown on cation adjusted Mueller Hinton Broth with 1.5% agar into liquid CAMHB. These were diluted 1:10,000 into CAMHB medium with respective treatment and grown at 37° C. with 225 rpm shaking. Optical density measurements were taking using a Tecan GENios at 562 nm with a bandwidth of 35 nm. Lag time was calculated using Growth Rates Made Easy (Hall et al. Mol. Biol. Evol. 31(1):232-238).

Colony Forming Unit Analysis.

Cultures were sampled at respective time points and serial dilutions were performed ranging from 10²-10¹⁰. For ampicillin sensitivity experiment, dilutions were plated on solid media and 300 μg/mL ampicillin sodium salt and grown at 37° C. for 24 hr followed by cell counting. For the other experiments, cultures were serially diluted into PBS and plated onto 2% agar and 1.5% agar. Plates were incubated for 16-24 h at 37° C. and counted for visible colonies.

Antisense Agents.

Newly designed PNAs were purchased from PNA Bio, Inc (Thousand Oaks, Calif.). PNA was re-suspended in 5% DMSO in water at 100 μM. Working stocks were stored at 4° C. and long term stocks at −20° C. to limit freeze thaw cycles.

Bla RNA Collection.

Three biological replicates were used for bla expression analysis. Cultures were pretreated overnight in respective PNA and liquid media in the absence of ampicillin and collected for RNA at 16 hr.

Mutant RNA Collection.

Three biological replicates from each mutant were chosen for stress response gene expression analysis. Mutant populations 1 and 2 were re-grown from respective freezer stocks in liquid media with 2.5 μM α-TSS and 300 μg/mL ampicillin at 37° C. with shaking. At 16 hr, 1:100 dilutions were plated onto solid media with 300 μg/mL ampicillin and grown at 37° C. for 16 hr. Individual colonies were selected and regrown in liquid media, 2.5 μM α-TSS, and 300 μg/mL ampicillin until they reached mid-log phase (OD 0.4-0.5). This method was used to sample individual biological replicates in the mutant populations.

RNA Extraction and Quantitative RT-PCR.

50 μL of the respective culture was added to 100 μL of Bacteria RNAprotect (Qiagen) and incubated at room temperature for 5 min. The cells were then pelleted via centrifugation for 3 min at 10,000 rpm, the supernatant removed, and samples were flash frozen in an ethanol dry ice bath and stored at −80° C. Precautions were taken to protect RNA from RNases using RNaseZap (Life Technologies). RNA was extracted from frozen cell pellets using GeneJET RNA purification kit (Thermo Scientific) and 350-600 ng was treated with Turbo DNA-free (Ambion). 50 ng cDNA was synthesized using Maxima Universal First Stand cDNA synthesis kit (Thermo Scientific). Primers for qPCR were purchased from Integrated DNA Technologies and are listed in Table 3. 1.5 ng of cDNA was used for qPCR with Maxima SYBR Green qPCR master mix with ROX normalization (Thermo Scientific) using Illumina Eco qPCR system. Transcript levels were analyzed using the ΔC_{q} method with respect to moderately expressed housekeeping gene cysG (43). Transcript levels were further analyzed for the mutant populations using the ΔΔC_{q} method with respect to the no treatment populations.

β-Lactamase Activity.

Fluorocillin™ Green 495/525 β-lactamase substrate soluble product (Life Technologies) was used at a concentration of 2.2 μM as a β-lactamase substrate and measured using a Tecan GENios microplate reader in black flat bottom 96 well plate at 485/535 nm with a bandwidth of 35 nm. Three biological replicates were grown from colonies for 12 hours liquid media in the presence of the respective antisense agents, diluted 1:10 into liquid media and Fluorocillin green, and monitored in the Tecan GENios for 5 hr at 37° C. measuring every 2 min. The slope of fluorescence measured was used as a measure of β-lactamase activity.

TABLE 3
Primers used for gene expression analysis qPCR. Product lengths were 150-200 nt
and the melting temperatures were ~60° C.

| Gene | | SEQ ID | | SEQ ID | Product |
|---|---|---|---|---|---|
| target | Forward Primer | NO: | Reverse Primer | NO: | Length (nt) |
| acrA | AAGCCCTTCTTCCAGACGTG | 19 | AACGGCAAAGCCAAAGTGTC | 20 | 189 |
| ampR | GCCTTCCTGTTTTTGCTCAC | 21 | ATAATACCGCGCCACATAGC | 22 | 186 |
| cyoA | TGGTAATGGGCTTCTCGTCG | 23 | TGGITTCGCCTGGAAGTACC | 24 | 197 |
| dinB | GGCCAGTTTGTGATTACGCC | 25 | CTACGCTCCCACAAAATGCG | 26 | 200 |
| Hfq | ATCCGTTCCTGAACGCACTG | 27 | ACTGTGATGAGAAACCGGGC | 28 | 191 |
| lexA | GTTAACGGCCAGGCAACAAG | 29 | TCAATAACGCCTTTGCGTGC | 30 | 162 |
| marA | AATCGCGCAAAAGCTGAAGG | 31 | GCGATTCGCCCTGCATATTG | 32 | 155 |
| mutS | ATGGAACGTGAGCAGGACAG | 33 | CAGCCAGCGTTTCAGCATAC | 34 | 156 |
| polB | GATCCAGCGTTGACCAAGTG | 35 | CGCCAGATACCATTTTGATGCG | 36 | 179 |
| recA | AGGGCGTCACAGATTTCCAG | 37 | GTAAAACCACGCTGACGCTG | 38 | 184 |
| Rob | ATCAGCGGCGTATCTTCCAG | 39 | ACCGCTTCGACTCTCAACAG | 40 | 178 |
| rpoS | AACGGCGGGCAATTTTTACC | 41 | AACTGTTATCGCAGGGAGCC | 42 | 195 |
| soxS | TCTGCTGCGAGACATAACCC | 43 | ACTTGCAACGAATGTTCCGC | 44 | 150 |
| tolC | ACGCACTACCACCAGTAACG | 45 | TTTGTCTTCCGGGACCAGTG | 46 | 189 |
| cysG | ATTCCGTTCTCGGTGGTTCC | 47 | CCAGCGTCTGTTTTTCTGCC | 48 | 172 |

Ampicillin Sensitivity Analysis.

Three biological replicates were selected from colonies and pretreated for 16 hr with respective α-TSS concentration in liquid media. After pretreatment, the cultures were diluted 1:100,000 into liquid media with respective concentration of ampicillin and α-TSS and allowed to grow with shaking at 37° C. for 24 hr. The final OD at 562 nm at 24 hr was used for data analysis.

Data Analysis.

Data are represented as mean±standard deviation. Single factor ANOVA was performed with confidence of p<0.05. Replicates shown are biological replicates.

Data Fitting.

Data fitting analysis was performed in Origin Pro 6.1. Data was fit to a sigmoidal/decay Boltzman function.

Clustering Analysis.

The coefficient of variation (COV) is defined as the standard deviation divided by the mean of the samples. The COV was calculated for the no treatment population (n=3), mutant population 1 (n=4), and mutant population 2 (n=3) using the ΔCt method with respect to cysG. The clustergram function (44) in the MATLAB Bioinformatics Toolbox (The Mathworks, Inc., Natick, Mass.) was used to perform hierarchical clustering of the COV's for gene expression analysis and to generate the heatmap and dendrogram. The standard setting of optimal leaf ordering, Euclidean pairwise distance calculation, and an unweighted average distance linkage function were used for the clustergram function.

### Example II. Targeting the Ribosomal Binding Site and Translation Start Site Reverts β-Lactamase Resistance

Protein functional sites on mRNA have been shown to be effective antisense sites for blocking ribosomal binding and migration. These include the ribosomal binding site (RBS) and translation start site (TSS), which are located in the 5′ untranslated region (UTR). The antisense oligomers disclosed and described herein are predicted to sterically hinder the ribosome. In order to prevent the production of truncated, but potentially active, β-lactamase enzyme, the 5′ UTR of bla mRNA was targeted (27).

Three different antisense oligomers were designed against target sites proximal to the 5′ UTR of bla mRNA: α-RBS; α-TSS; and α-YUNR (FIGS. 1B and 6) (28). Antisense oligomers were designed with the target sequence in the middle of the oligomer, with 3-5 nucleotides flanking the overlapping region (27). To prevent translation of β-lactamase, two short 12-mer antisense oligomers—α-RBS (C-ATAACTTTTTCC-N(SEQ ID NO: 1); RBS in bold) and α-TSS (C-TCTCATACTCAT-N(SEQ ID NO: 3); translational start site in bold)—were designed against the ribosomal binding site and translational start site, respectively (FIGS. 1B and 6). While α-RBS was designed to prevent the ribosomal binding, α-TSS was designed to prevent ribosomal migration, both causing inhibition of translation of bla transcript.

The third antisense agent, α-YUNR (C-AGCGGGAATAAG-N(SEQ ID NO: 5); YUNR in bold), was designed to target the YUNR sequence motif on the stem loop between nucleotides 61-78 of bla transcript (FIGS. 1B and 6). The YUNR motif (pyrimidine, uracil, any ribose nucleic acid, and a purine) has been shown to have high antisense binding affinity due to formation of intraloop hydrogen bonds facilitating a U-turn structure, and is known to initiate rate limiting interactions in a number of naturally occurring systems (29, 30). The targeted YUNR motif was represented in a single stranded region of a stem loop in 14/19 free energy secondary structures of the bla RNA modeled using RNAstructure software (FIG. 1A). α-YUNR was also designed to prevent ribosomal migration, thus inhibiting translation of bla to β-lactamase enzyme.

To prevent degradation of antisense oligomers by endonucleases expressed by the host cell, non-natural antisense PNA oligomers were used. PNA has a modified peptide backbone with nucleic acid functional groups and exhibits no known enzymatic cleavage, which leads to increased stability in cells (31). PNA molecules undergo Watson-Crick base paring with RNA and DNA, thereby enabling antisense interaction (19). Additionally, PNAs have higher binding affinity and form more stable interactions with RNA and DNA than natural nucleic acids due to a neutral backbone (19). Further, since PNA is a non-natural molecule, bacteria is less likely to have an inherent resistance mechanism against the molecule. The α-RBS, α-TSS, and α-YUNR PNA molecules were designed as 12mers for increased affinity to the target site (32). The 12mers were conjugated to an O-linker for reduced steric interference in target binding, as well as to a cell penetrating peptide (KFF)₃K (SEQ ID NO: 49), for increased transport into gram-negative bacteria cells (32) (Table 1). The 12mer antisense sequences were searched against the E. coli K-12 genome 133 (U00096.2) using NCBI BLAST to evaluate target selectivity and to avoid off-target interactions. α-RBS, α-TSS, and α-YUNR searches returned no matches to the E. coli K-12 genome (33).

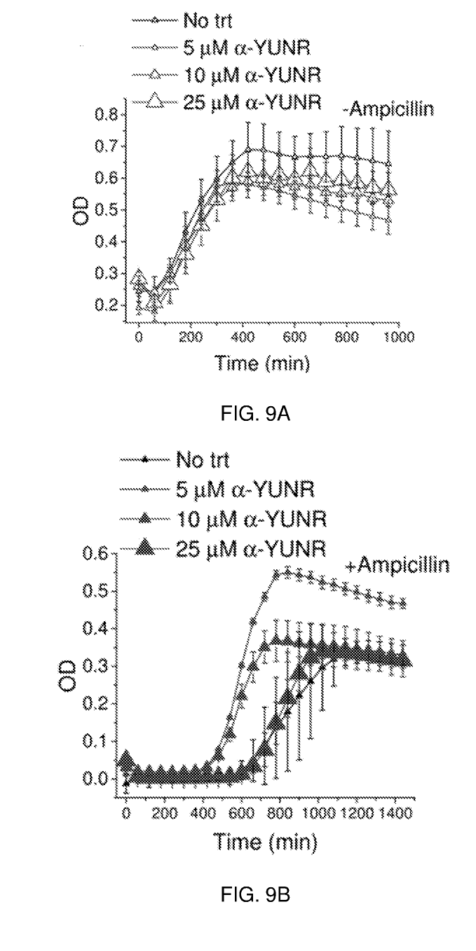

α-RBS, α-TSS, and α-YUNR were tested to identify a minimum inhibitory concentration (MIC) between 1-25 μM, based on concentrations reported in previous studies conducted in E. coli for CPP conjugated PNA (27, 31, 32, 34). E. coli cultures, pretreated overnight with respective antisense agents in the absence of ampicillin, grew similarly to untreated cells, implying lack of toxicity of the antisense agents (FIGS. 7A, 8A, and 9A). In the presence of ampicillin and α-TSS at a MIC of 2.5 μM, the growth rate of ampicillin-resistant E. coli was significantly inhibited (p<0.05) (FIGS. 2 and 7B). Inhibition of growth of drug resistant E. coli was observed in presence of ampicillin and an elevated MIC of 25 μM for α-RBS (p<0.05) (FIGS. 2 and 8B). α-YUNR did not show growth inhibition up to 25 μM (p>0.05) (FIGS. 2 and 9B). α-TSS and α-RBS re-sensitized drug resistant E. coli to ampicillin and hindered cell growth, only in the presence of ampicillin, indicating gene specific targeting of bla gene as well as non-toxic effect of the RNA inhibitors in absence of ampicillin.

### Example III. Translational Inhibition Mechanism of Action of α-RBS and α-TSS Antisense RNA Inhibitors

Using quantitative real-time polymerase chain reaction (qPCR), expression levels of the bla gene in presence of the three antisense agents were measured. Studies were carried out with 5 μM α-TSS, 25 μM α-RBS, and 25 μM α-YUNR in the absence of ampicillin. RNA expression analysis of bla transcript showed similar levels of bla RNA both in absence and presence of treatment with the antisense agents (p>0.05) (FIG. 3A), indicating that the antisense agents did not inhibit the expression of bla transcript. To evaluate the impact of the antisense agents on translation of the bla gene, a β-lactamase activity assay was used to measure β-lactamase protein activity. α-TSS and α-RBS significantly reduced β-lactamase activity (p<0.05) (FIG. 3B). α-YUNR had no impact on protein activity (p>0.05) (FIG. 3B). These results demonstrate that α-TSS and α-RBS reduced β-lactamase activity, but did not affect bla transcript levels, thus indicating that α-TSS and α-RBS act via a translational inhibition mechanism; by blocking the translation start site and ribosomal binding site, respectively, to prevent translation to β-lactamase protein. These results are consistent with the growth behavior shown in FIG. 2 and FIGS. 7-9, where α-TSS and α-RBS impact growth of drug-resistant E. coli in presence of ampicillin and α-YUNR had no impact on cell growth.

### Example IV. α-TSS RNA Inhibitor Restores Drug-Sensitivity

To evaluate the therapeutic potential of RNA inhibitors, the best-performing antisense agent α-TSS was used. Overnight cultures of ampicillin-resistant E. coli were pre-treated with different concentrations of α-TSS, followed by treatment with ampicillin. Since α-TSS inhibits β-lactamase production, it was hypothesized that α-TSS would restore the bactericidal effect of ampicillin Indeed, α-TSS decreased cell viability at the MIC of 2.5 μM and higher by at least 1000 fold within the first three hours of treatment with ampicillin (FIGS. 4A-4B). Below the MIC of α-TSS (no treatment case and 1 μM α-TSS), colony forming units increased with time (FIG. 4A).

The degree of re-sensitization exerted by α-TSS in presence of varying concentrations of ampicillin above and below the MIC determined for α-TSS was then evaluated. Three concentrations of α-TSS were tested: no treatment, 0.5 μM α-TSS (below MIC), and 5 μM α-TSS (above MIC) (See Materials and Methods) (FIG. 4C). In absence of treatment, ampicillin-resistant E. coli was able to grow in up to 300 μg/mL ampicillin without inhibition, and showed a decrease in growth until 700 μg/mL ampicillin, where no growth was observed. At below MIC level of 0.5 μM, α-TSS ampicillin-resistant E. coli grew similar to the no treatment case with unhindered growth up to 250 μg/mL ampicillin and no growth at 550 μg/mL. At 5 μM α-TSS (above MIC), ampicillin-resistant E. coli only grew unhindered without ampicillin and showed a decrease in growth as low as 25 μg/mL, which is a greater than 20 fold decrease in ampicillin MIC compared to the no treatment cultures. Data fitting analysis (see Materials and methods) on the drug-sensitivity curves showed that the slope of the transition state, from resistant to sensitive, is altered by α-TSS. The slope of the transition for no treatment was −0.0051 ODmLμg⁻¹ and for below MIC (0.5 μM) was −0.0038 ODmLμg⁻¹. For above MIC (5 μM) the slope was −0.010 OD/μg/mL. Greater than two fold increase in negative slope for above MIC compared to below MIC and no treatment indicates that while the α-TSS is re-sensitizing the E. coli to ampicillin, it is changing the sensitivity landscape with respect to the resistant strain (FIG. 4C).

### Example V. Adaptive Resistance to α-Bla RNA Inhibitor Demonstrates Gene Expression Heterogeneity

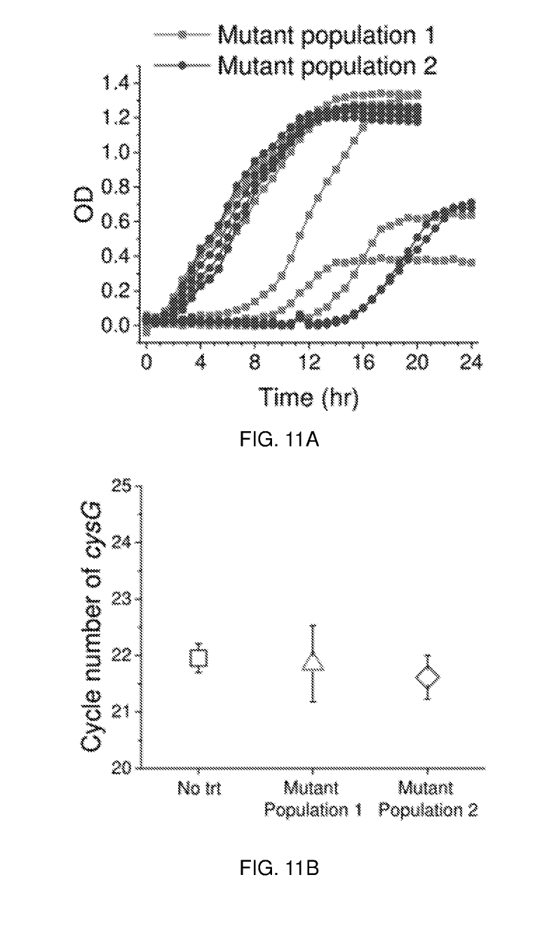

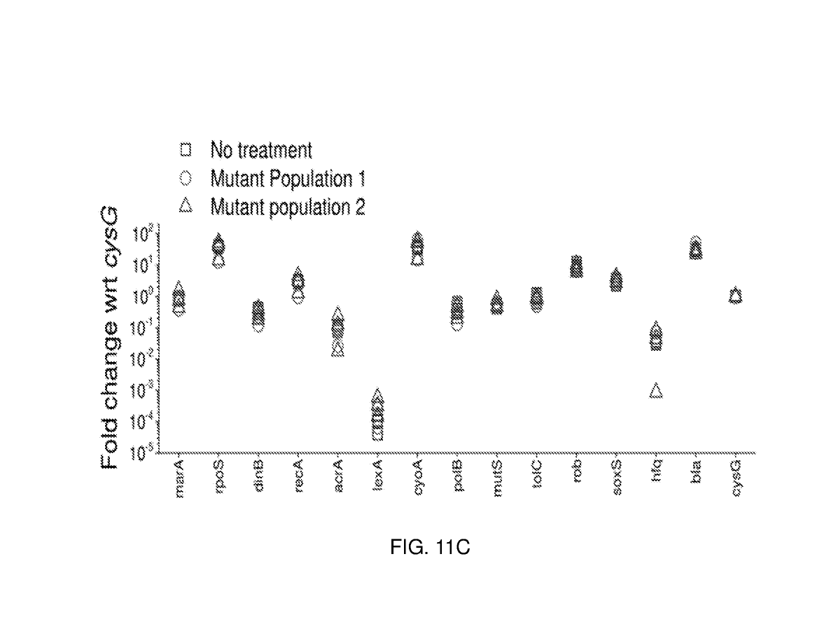

Since resistance has been reported for enzyme based β-lactamase inhibitors (8), α-TSS was evaluated at the MIC to investigate the emergence of resistance. Ampicillin-resistant cultures were pretreated overnight with 2.5 μM α-TSS and then subjected to selection pressure of 2.5 μM α-TSS and 300 μg/mL of ampicillin for 24 hours. In a set of 35 independent cultures, only two cultures developed tolerance to ampicillin/α-TSS combination in 24 hours, hereby referred to as mutant populations 1 and 2 (FIG. 10A). Mutant populations 1 and 2 were diluted and re-grown under selection pressure for another 24 hours. The mutants grew, confirming that they were stable mutants that had developed resistance to α-TSS and that their growth was not an artifact of ampicillin or α-TSS degradation (FIG. 10B). The biological mutant replicates showed high variability in growth rate compared to the no treatment biological replicates (FIGS. 5A and 11A). Within the mutant populations, the growth rates varied between 0.08-0.14 hr⁻¹ for mutant population 1 and 0.09-0.16 hr⁻¹ for mutant population 2. The heterogeneous growth behavior of mutant colonies indicated the presence of alternate mechanisms of adaptive resistance.

None (out of 35) of the independent cultures grown at the 5μM α-TSS/β-lactam showed emergence of tolerance to the therapeutic combination. When cultures were exposed to lower (MIC) concentration of 2.5μM α-TSS/β-lactam, less than 5% of the independent cultures (2 out of 35) grown at the MIC showed emergence of tolerance to the β-lactam/α-TSS therapeutic combination. Changes in expression of a set of key stress response genes was examined to identify the resistance mechanism involved in obtaining resistance to α-TSS. Thirteen representative stress response genes were measured by qPCR (FIG. 5B). By quantifying mRNA expression of these representative stress response genes, gene expression heterogeneity was shown to play a role in adaptive tolerance to the β-lactam/α-TSS combination in the mutant populations. Using hierarchical clustering, mutant populations can be clustered based on the COV of gene expression. Compared to no treatment case, 10 out of the 13 stress-response genes in mutant population 1, and 12 out of 13 stress-response genes in mutant population 2 demonstrated significant changes in the COV when compared to the no treatment case. marA, a gene activating broad-range efflux, had the greatest increase in variability compared to the no treatment population.

### Example VI. Additional Antisense Inhibitors

Having demonstrated the ability of α-bla antisense inhibitors to re-sensitize drug resistant E. coli to ampicillin, six additional antisense inhibitors were designed to target other genes (Table 2). Similarly to the α-bla antisense inhibitors, PNA oligomers were used to prevent degradation of the antisense oligomers by endonucleases. PNA molecules targeting folC involved in metabolism, ffh which is a part of the signal recognition particle, lexA a key regulator of stress response, and fnrS, a small Hfq binding RNA were designed and generated. Two PNA molecules against essential genes in traditional antibiotic pathways for rpsD involved in protein biosynthesis and gyrB involved in DNA replication were also generated. These two genes were chosen to mimic current antibiotic in traditional pathways and to investigate synergistic and antagonistic antibiotic interactions. Although the PNA molecules were designed against essential genes in E. coli, potential off targets of the PNA in E. coli were also investigated, as well as homology in two other bacteria, Klebsiella pneumoniae and Salmonella enterica. The molecules were designed to have homology to all three organisms where possible so that the sequence-specific oligomers could be applied to target multiple pathogenic bacteria highlighting the potential to create broad-spectrum gene specific antibiotics. Oligomers were also designed to target only an essential gene in E. coli, one which had one off target, and one that had three off targets to investigate off target potential of PNA antibiotics. The additional antisense oligomers were 12 nucleotides long and centered around the start codon of each target gene. With this arsenal of non-conventional antibiotics, the effectiveness of combinations of PNA molecules and combinations of PNA with conventional antibiotics was studied.

### Example VII. Antisense Inhibitors Targeting Novel Pathways Inhibit Growth of E. coli

Cultures of MG1655 E. coli were completely inhibited for 24 h by each of 10 μM of α-lexA, α-fnrS, and α-rpsD (FIGS. 13A-13C). The lag time was significantly increased by treatment with α-lexA, α-fnrS, α-rpsD, and α-gyrB. The effect of α-lexA, α-fnrS, and α-rpsD on cell viability using a colony forming unit analysis was then determine. 10 μM α-rpsD was found to reduce colony forming units per milliliter to zero after only two hours of treatment highlighting its bactericidal effect (FIGS. 13D-13E). In addition, α-lexA prevented significant increase in viable cells at six and eight hours, highlighting its bacteriostatic effect on cell growth (FIGS. 13D-13E).

### Example VIII. Antisense Inhibitors Targeting Novel Pathways Inhibit Growth of Clinically Isolated Multi Drug Resistant Bacteria

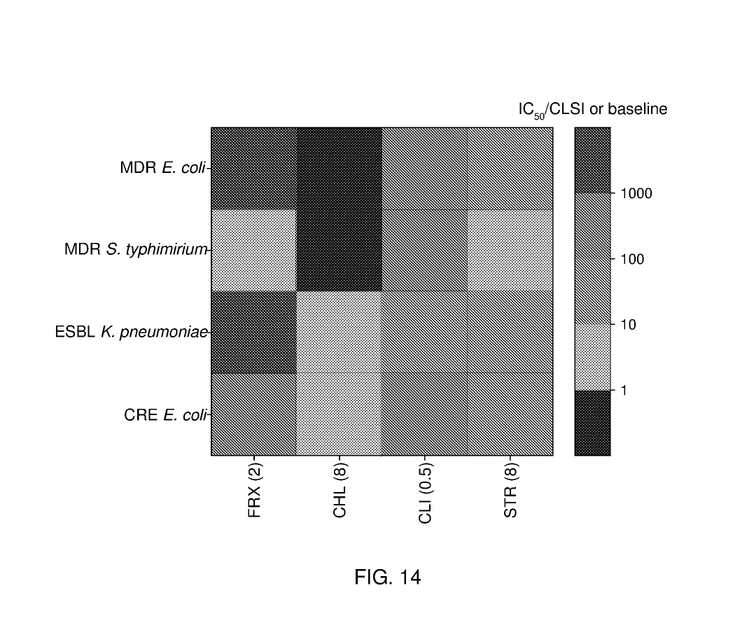

For highly resistant clinical strains, antisense therapeutics were shown to target novel, non-traditional antibiotic targets and functioned both alone and in combination with conventional antibiotics (FIGS. 14-17). Antisense therapeutics were shown to be gene specific, yet still possess broad-spectrum over a range of clinical gram-negative pathogens (FIGS. 15-16). Multi drug resistant (MDR) S. typhimurium and carbapenem-resistant enterobacteriaceae (CRE) E. coli were shown to be inhibited by all six oligomers tested (FIGS. 15-16). MDR E. coli was inhibited by four of the six oligomers tested (α-folC, α-ffh, α-lexA, and α-rpsD), extended-spectrum beta-lactamase (ESBL) K. pneumoniae was inhibited by two of the antisense oligomers (α-fnrS and α-rpsD), and New Delhi metallo-β-lactamase (NDM-1) K. pneumoniae was inhibited by one of the oligomers tested (α-rpsD) (FIGS. 15-16). The predicted sequence homology was examined in lab stains of the bacterium, and were shown to function in the clinical pathogens without knowledge of the sequence of the pathogens. Of those antisense oligomers tested, α-rpsD was identified as the best functioning oligomer against all strains tested, and completely prevented growth of CRE E. coli (FIGS. 15-16).

### Example IX. Antisense Inhibitors can be Used in Combination with Conventional Antibiotics as “Resistance-Breakers” for Treating Multi Drug Resistant Bacteria

To highlight the utility of the antisense oligomers targeting novel pathways, combinations of α-gyrB with ciprofloxacin or chloramphenicol were examined. Ciprofloxacin is a small molecule antibiotic which targets DNA gyrase, while chloramphenicol is a small molecule antibiotic which targets the 23s rRNA of the 50s ribosomal subunit. α-gyrB was combined with ciprofloxacin to target the same target in one pathway (FIG. 17A), or with chloramphenicol to target multiple pathways and different targets (FIG. 17A). The combination of antisense therapeutics and antibiotics was shown to be effective on clinical strains (FIGS. 17B-17C).

### Example X. Peptide Nucleic Acid-Based Antisense Inhibitors are Non-Toxic to Mammalian Cells

Antisense therapeutics, specifically peptide nucleic acids, were shown to be non-toxic to HEK 293T cells, highlighting their usefulness as specific antibiotics (FIG. 18). They can not only be designed in sequence to prevent binding to mammalian sequences, but can also be conjugated to (KFF)₃K (SEQ ID NO: 49) peptides to increase their uptake into gram-negative cells.

### REFERENCES

(1) Center for Disease Control, States U. 2013. Antibiotic Resistance Threats in the United States, 2013.
(2) Yigit H, et al. 2001. Novel Carbapenem-hydrolyzing beta-lactamase, KPC-1, from a carbapenem-resistant strain of Klebsiella pneumoniae. Antimicrob. Agents Chemother, 45(4):1151-61.
(3) Daley C L and Horsburgh C R. 2014 (July). Treatment for Multidrug-Resistant Tuberculosis: It's Worse Than We Thought! Clin Infect Dis, 1-2. ePub Jul. 23, 2014.
(4) Hendriksen R S, et al. 2013. Extremely drug resistant Salmonella enterica serovar Senftenberg infections in patients in Zambia. J. Clin. Microbiol. 51:284-6.
(5) World Health Organization. 2012. Global Tuberculosis Report.
(6) Bush K, and Jacoby G. 2010. Updated functional classification of beta-lactamases. Antimicrob. Agents Chemother. 54:969-76.
(7) Bush K. 2013. Proliferation and significance of clinically relevant β-lactamases. Ann. N.Y. Acad. Sci. 12(77):84-90.
(8) Drawz S M, Papp-Wallace K M, and Bonomo R. 2014. New β-lactamase inhibitors: a therapeutic renaissance in an MDR world. Antimicrob. Agents Chemother. 58:1835-46.
(9) Livermore D M. 1998. Beta-lactamase-mediated resistance and opportunities for its control. J. Antimicrob. Chemother. 41 Suppl D:25-41.
(10) Shlaes D M. 2013. New β-lactam-β-lactamase inhibitor combinations in clinical development. Ann. N. Y. Acad. Sci. 1277:105-14.
(11) Queenan A M, Bush K. 2007. Carbapenemases: the versatile beta-lactamases. Clin. Microbiol. Rev. 20:440-58, table of contents.
(12) Brink A J, et al. 2012. Emergence of New Delhi metallo-beta-lactamase (NDM-1) and Klebsiella pneumoniae carbapenemase (KPC-2) in South Africa. J. Clin. Microbiol. 50:525-7.
(13) Chatterjee A, et al. 2013. Antagonistic self-sensing and mate-sensing signaling controls antibiotic-resistance transfer. Proc. Natl. Acad. Sci. U.S.A 110:7086-90.
(14) Mwangi M M, et al. 2007. Tracking the in vivo evolution of multidrug resistance in Staphylococcus aureus by whole-genome sequencing. Proc. Natl. Acad. Sci. U.S.A 104:9451-6.
(15) Bjedov I, et al. 2003. Stress-induced mutagenesis in bacteria. Science 300:1404-9.
(16) Davies J, and Davies D. 2010. Origins and evolution of antibiotic resistance. Microbiol. Mol. Biol. Rev. 74:417-33.
(17) Walsh C. 2000. Molecular mechanisms that confer antibacterial drug resistance. Nature 406:775-81.
(18) Bennett C F, and Swayze E E. 2010. RNA targeting therapeutics: molecular mechanisms of antisense oligonucleotides as a therapeutic platform. Annu. Rev. Pharmacol. Toxicol. 50:259-93.
(19) Egholm M, et al. 1993. PNA hybridizes to complementary oligonucleotides obeying the Watson-Crick hydrogen-bonding rules. Nature 365.
(20) Johnson E, and Srivastava R. 2013. Volatility in mRNA secondary structure as a design principle for antisense. Nucleic Acids Res. 41:e43.
(21) Massé E, Escorcia F E, and Gottesman S. 2003. Coupled degradation of a small regulatory RNA and its mRNA targets in Escherichia coli. Genes Dev. 17:2374-83.
(22) Zorzi F, et al. 2013. Smad7 antisense oligonucleotide-based therapy for inflammatory bowel diseases. Dig. Liver Dis. 45:552-5.
(23) Hnik P, et al. 2009. Antisense oligonucleotide therapy in diabetic retinopathy. J. Diabetes Sci. Technol. 3:924-30.
(24) Merki F, et al. 2008. Antisense oligonucleotide directed to human apolipoprotein B-100 reduces lipoprotein(a) levels and oxidized phospholipids on human apolipoprotein B-100 particles in lipoprotein(a) transgenic mice. Circulation 118:743-53.
(25) Zhang H, et al. 1999. Antisense Oligonucleotide Inhibition of Hepatitis C Virus (HCV) Gene Expression in Livers of Mice Infected with an HCV-Vaccinia Virus Recombinant Antisense Oligonucleotide Inhibition of Hepatitis C Virus (HCV) Gene Expression in Livers of Mice Infecte.
(26) Lu X, et al. 2004. Antisense-Mediated Inhibition of Human Immunodeficiency Virus (HIV) Replication by Use of an HIV Type 1-Based Vector Results in Severely Attenuated Mutants Incapable of Developing Resistance 78:7079-7088.
(27) Dryselius R, et al. 2003. The translation start codon region is sensitive to antisense PNA inhibition in Escherichia coli. Oligonucleotides 13:427-33.
(28) McAdams H H, and Arkin a. 1997. Stochastic mechanisms in gene expression. Proc. Acad. Sci. U.S.A 94:814-9. Natl.
(29) Franch T, et al. 1999. Antisense RNA regulation in prokaryotes: rapid RNA/RNA interaction facilitated by a general U-turn loop structure. J. Mol. Biol. 294:1115-25.
(30) Brunel C, et al. 2002. RNA loop-loop interactions as dynamic functional motifs. Biochimie 84:925-44.
(31) Hatamoto M, Ohashi A, and Imachi H. 2010. Peptide nucleic acids (PNAs) antisense effect to bacterial growth and their application potentiality in biotechnology. Appl. Microbiol. Biotechnol. 86:397-402.
(32) Good L, et al. 2001. Bactericidal antisense effects of peptide-PNA conjugates. Nat. Biotechnol. 19:360-4.
(33) Wang J, et al. 1997. Mismatch-sensitive hybridization detection by peptide nucleic acids immobilized on a quartz crystal microbalance. Anal. Chem. 69:5200-2.
(34) Good L, and Nielsen P E. 1998. Antisense inhibition of gene expression in bacteria by PNA targeted to mRNA. Nat. Biotechnol. 16.
(35) Magnet S, et al. 2003. Aminoglycoside Resistance Resulting from Tight Drug Binding to an Altered Aminoglycoside Acetyltransferase. Antimicrob. Agents Chemother. 47:1577-1583.
(36) Triglia T, et al. 1997. Mutations in dihydropteroate synthase are responsible for sulfone and sulfonamide resistance in Plasmodium falciparum. Proc. Natl. Acad. Sci. U.S.A 94:13944-9.
(37) Sommer M O, and Dantas G. 2011. Antibiotics and the resistant microbiome. Curr. Opin. Microbiol. 14:556-63.
(38) Ruden M, and Puri N. 2013. Novel anticancer therapeutics targeting telomerase. Cancer Treat. Rev. 39:444-56.
(39) Fan X-K, et al. 2014. Antisense oligodeoxynucleotide against human telomerase reverse transcriptase inhibits the proliferation of Eca-109 esophageal carcinoma cells. Exp. Ther. Med. 8:1247-1252.
(40) Kole R, KraMer A R, and Altman S. 2012. RNA therapeutics: beyond RNA interference and antisense oligonucleotides. Nat. Rev. Drug Discov. 11:125-40.
(41) Montgomery R L, et al. 2011. Therapeutic inhibition of miR-208a improves cardiac function and survival during heart failure. Circulation 124:1537-47.
(42) Karsi A, and Lawrence M L. 2007. Broad host range fluorescence and expression vectors for Gram-negative bacteria. Plasmid 57:286-95. bioluminescence
(43) Zhou A., et al. 2011. Novel reference genes for quantifying transcriptional responses of Escherichia coli to protein overexpression by quantitative PCR. BMC Mol. Biol. 12:18.
(44) Bar-joseph Z, Gifford D K, and Jaakkola T S. 2001. Fast optimal leaf ordering for hierarchical clustering. Bioinformatics 17 Suppl1:S22-9.

While the invention has been described with reference to various and preferred embodiments, it should be understood by those skilled in the art that various changes can be made and equivalents can be substituted for elements thereof without departing from the essential scope of the invention. In addition, many modifications can be made to adapt a particular situation or material to the teachings of the invention without departing from the essential scope thereof.

Therefore, it is intended that the invention not be limited to a particular embodiment disclosed herein contemplated for carrying out this invention, but that the invention will include all embodiments falling within the scope of the claims.

What is claimed is:

1. An antisense antibiotic oligomer comprising a nucleic acid sequence complementary to at least one target selected from the group of: at least one target site on a DNA sequence of an essential bacterial gene associated with an antibiotic pathway; at least one target site on a DNA sequence of a bacterium associated with antibiotic resistance; at least one target site on an RNA sequence of the bacterium associated with antibiotic resistance; at least one target site on an mRNA sequence of the bacterium which encodes a protein essential for bacterial homeostasis; and at least one target site on an mRNA sequence of the bacterium which encodes a protein associated with antibiotic resistance.

2. The antisense antibiotic oligomer of claim 1, wherein the antisense antibiotic oligomer is RNA or a nucleic acid analog chosen from the group of: peptide nucleic acid oligomer; morpholino oligomer; locked nucleic acid oligomer; bridged nucleic acid oligomer; and 2′-O-methyl-substituted RNA oligomer.

3. The antisense antibiotic oligomer of claim 1, wherein the nucleic acid sequence complementary to the at least one target has a sequence length selected from the group of: about 5-mers to about 40-mers; about 10-mers to about 15-mers; and 12-mers.

4. The antisense antibiotic oligomer of claim 1, further comprising a cell penetrating peptide conjugated to the nucleic acid sequence complementary to the at least one target.

5. The antisense antibiotic oligomer of claim 4, wherein the cell penetrating peptide is selected from the group of: (KFF)₃K (SEQ ID NO: 49); penetratin; NLS; TAT; Arg(9); D-Arg(9); 10HC; cyLoP-1; and Pep-1.

6. The antisense antibiotic oligomer of claim 4, wherein the cell penetrating peptide is conjugated to the nucleic acid sequence via a linker.

7. The antisense antibiotic oligomer of claim 6, wherein the linker is selected from the group of: O-linker; E-linker; C6A linker; C6SH linker; X-linker; and C11SH linker.

8. The antisense antibiotic oligomer of claim 1, wherein the nucleic acid sequence complementary to at least one target site on a DNA sequence is centered around a start codon.

9. The antisense antibiotic oligomer of claim 1, wherein the antibiotic pathway is selected from the group of: DNA replication and cell growth; protein biosynthesis; cell wall synthesis; transport and membrane function or bio synthesis; metabolism; redox homeostasis, stress response, and cell signaling; replication and growth; transcription and translation; and DNA modification, repair, and maintenance.

10. The antisense antibiotic oligomer of claim 9, wherein the antibiotic pathway is transport and membrane function or bio synthesis and the essential bacterial gene is selected from the group of: folC; cdsA; msbA; lptA; sgrT; secA; secD; secE; secF; secM; and secY.

11. The antisense antibiotic oligomer of claim 10, wherein the essential bacterial gene is folC and the complementary nucleic acid sequence has the sequence of SEQ ID NO: 7.

12. The antisense antibiotic oligomer of claim 9, wherein the antibiotic pathway is metabolism and the essential bacterial gene is selected form the group of: adk; coaD; eno; ispA; ispB; ispD; ispE; ispF; ispG; ispH; and ispU.

13. The antisense antibiotic oligomer of claim 9, wherein the antibiotic pathway is redox homeostasis, stress response, and cell signaling and the essential bacterial gene is selected form the group of: can; grpE; lexA; rseP; rpoE; ffh; ffs; lepB; and ispA.

14. The antisense antibiotic oligomer of claim 13, wherein the essential bacterial gene is ffh and the complementary nucleic acid sequence has the sequence of SEQ ID NO: 9.

15. The antisense antibiotic oligomer of claim 13, wherein the essential bacterial gene is lexA and the complementary nucleic acid sequence has the sequence of SEQ ID NO: 11.

16. The antisense antibiotic oligomer of claim 9, wherein the antibiotic pathway is replication and growth and the essential bacterial gene is selected form the group of: era; odgE; ftsA; ftsB; ftsE; ftsI; ftsK; ftsL; ftsQ; ftsW; ftsZ; holA; and holB.

17. The antisense antibiotic oligomer of claim 9, wherein the antibiotic pathway is transcription and translation and the essential bacterial gene is selected form the group of: bamA; bamD; gyrA; gyrB; prfA; rpsA; rpsB; rpsC; rpsD; rpsE; rpsH; rpsJ; rpsK; rpsL; rpsN; rpsP; rpsR; and rpsS.

18. The antisense antibiotic oligomer of claim 17, wherein the essential bacterial gene is gyrB and the complementary nucleic acid sequence has the sequence of SEQ ID NO: 15.

19. The antisense antibiotic oligomer of claim 17, wherein the essential bacterial gene is rpsD and the complementary nucleic acid sequence has the sequence of SEQ ID NO: 17.

20. The antisense antibiotic oligomer of claim 9, wherein the antibiotic pathway is DNA modification, repair, and maintenance and the essential bacterial gene is selected form the group of: ligA; prmC; and trmD.

21. The antisense antibiotic oligomer of claim 1, wherein the essential bacterial gene is selected from the group of: fnrS; ilvX; apbE; nusA; rpoD; nusE; ffh; rpsU; accD; degS; ftsN; lolA; hflB; mraY; rsG; rplV; nadD; murF; murA; and mreD.

22. The antisense antibiotic oligomer of claim 21, wherein the essential bacterial gene is fnrS and the complementary nucleic acid sequence has the sequence of SEQ ID NO: 13.

23. The antisense antibiotic oligomer of claim 1, wherein the at least one target site on the mRNA sequence is selected from the group of: a translation start site; a ribosomal binding site; and a YUNR motif.

24. The antisense antibiotic oligomer of claim 1, wherein the mRNA sequence of the bacterium is transcribed from a gene bla and the complementary nucleic acid sequence is bla α-RBS (SEQ ID NO: 1) or bla α-TSS (SEQ ID NO: 3).

25. The antisense antibiotic oligomer of claim 1, wherein the antisense antibiotic oligomer is a peptide nucleic acid conjugated to a (KFF)₃K (SEQ ID NO: 49) cell penetrating peptide via an O-linker.

26. The antisense antibiotic oligomer of claim 1, wherein the antisense antibiotic oligomer has a sequence selected from the group of: SEQ ID NO: 2; SEQ ID NO: 4; SEQ ID NO: 8; SEQ ID NO: 10; SEQ ID NO: 12; SEQ ID NO: 14; SEQ ID NO: 16; and SEQ ID NO: 18.

27. An antibiotic composition comprising at least one antisense antibiotic oligomer of any one of claims 1-26.

28. The antibiotic composition of claim 27, wherein the antibiotic composition comprises two or more antisense antibiotic oligomers and a first antisense antibiotic oligomer targets a first target and a second antisense antibiotic oligomer targets a second target different from the first target.

29. The antibiotic composition of claim 28, wherein the first target and the second target are located on the same DNA sequence, RNA sequence, or mRNA sequence.

30. The antibiotic composition of claim 28, wherein the first target and the second target are located on different DNA sequences, RNA sequences, or mRNA sequences.

31. The antibiotic composition of claim 30, wherein a first mRNA sequence and a second mRNA sequence each encode a unique protein associated with antibiotic resistance, a unique protein essential for bacterial homeostasis, or a first mRNA sequence encoding a protein associated with antibiotic resistance and a second mRNA sequence encoding a protein essential for bacterial homeostasis.

32. The antibiotic composition of claim 28, wherein the first target is located on a DNA sequence and the second target is located on an mRNA sequence.

33. The antibiotic composition of claim 27, wherein the antibiotic composition comprises 10 or more antisense antibiotic oligomers.

34. The antibiotic composition of claim 27, further comprising at least one conventional antibiotic.

35. The antibiotic composition of claim 34, wherein the at least one conventional antibiotic is selected from the group of: penicillins; cephalosporins; carbacephems; cephamycins; carbapenems; monobactams; aminoglycosides; glycopeptides; quinolones; tetracyclines; macrolides; and fluoroquinolones.

36. The antibiotic composition of claim 34, wherein the at least one conventional antibiotic targets the same target of the same pathway as the at least one antisense antibiotic oligomer, a different target of the same pathway as the at least one antisense antibiotic oligomer, or a different target in a different pathway than the at least one antisense antibiotic oligomer.

37. The antibiotic composition of claim 34, wherein the at least one antisense antibiotic oligomer comprises at least one of bla α-RBS (SEQ ID NO: 1) and bla α-TSS (SEQ ID NO: 3), and the at least one conventional antibiotic is a β-lactam.

38. The antibiotic composition of claim 37, further comprising a pharmaceutical β-lactamase inhibitor.

39. The antibiotic composition of claim 27, wherein the antibiotic composition is a pharmaceutical composition.

40. The antibiotic composition of claim 34, wherein the at least one antisense antibiotic oligomer comprises α-gyrB (SEQ ID NO: 15) and the conventional antibiotic is chloramphenicol or ciprofloxacin.

41. A method for re-sensitizing a subject to one or more conventional antibiotics in need thereof, comprising administering to the subject a pharmaceutically effective amount of a composition of claim 27 wherein the at least one antisense antibiotic oligomer targets at least one of a DNA sequence, RNA sequence, or mRNA sequence associated with antibiotic resistance.

42. The method of claim 41, wherein the at least one antisense antibiotic comprises at least one of bla α-RBS (SEQ ID NO: 1) and bla α-TSS (SEQ ID NO: 3) and the conventional antibiotic is a β-lactam.

43. The method of claim 41, wherein the subject is selected from the group of: human; cattle; swine; poultry; goat; sheep; dog; cat; rodent; bird; and reptile.

44. A method for treating a bacterial infection in a subject in need thereof, comprising re-sensitizing a subject to one or more conventional antibiotics by administering to the subject a pharmaceutically effective amount of a composition of claim 27 wherein the at least one antisense antibiotic oligomer targets at least one of a DNA sequence, RNA sequence, mRNA sequence associated with antibiotic resistance and administering to the subject at least one antibiotic to which the subject has been re-sensitized.

45. The method of claim 44, wherein the subject is administered a composition comprising at least one of bla α-RBS (SEQ ID NO: 1) and bla α-TSS (SEQ ID NO: 3), and the at least one conventional antibiotic is a β-lactam.

46. The method of claim 45, wherein the composition further comprises a pharmaceutical β-lactamase inhibitor.

47. The method of claim 44, wherein the subject is selected from the group of: human; cattle; swine; poultry; goat; sheep; dog; cat; rodent; bird; and reptile.

48. A method for treating a bacterial infection in a subject in need thereof, comprising administering to the subject a pharmaceutically effective amount of a composition of claim 27 wherein the at least one antisense antibiotic oligomer is bactericidal or bacteriostatic.

49. The method of claim 48, wherein the at least one antisense antibiotic oligomer comprises an a complementary nucleic acid sequence to at least one essential bacterial gene selected from the group of: folC; cdsA; msbA; lptA; sgrT; secA; secD; secE; secF; secM; secY; adk; coaD; eno; ispA; ispB; ispD; ispE; ispF; ispG; ispH; ispU; can; grpE; lexA; rseP; rpoE; ffh; ffs; lepB; lspA; era; odgE; ftsA; ftsB; ftsE; ftsI; ftsK; ftsL; ftsQ; ftsW; ftsZ; holA; holB; bamA; bamD; gyrA; gyrB; prfA; rpsA; rpsB; rpsC; rpsD; rpsE; rpsH; rpsJ; rpsK; rpsL; rpsN; rpsP; rpsR; rpsS; ligA; prmC; trmD; fnrS; ilvX; apbE; nusA; rpoD; nusE; ffh; rpsU; accD; degS; ftsN; lolA; hflB; mraY; rsG; rplV; nadD; murF; murA; and mreD.

50. The method of claim 48, wherein the at least one antisense antibiotic oligomer comprises an a complementary nucleic acid sequence to at least one essential bacterial gene selected from the group of: folC wherein the complementary nucleic acid sequence has the sequence of SEQ ID NO: 7; ffh wherein the complementary nucleic acid sequence has the sequence of SEQ ID NO: 9; lexA wherein the complementary nucleic acid sequence has the sequence of SEQ ID NO: 11; gyrB wherein the complementary nucleic acid sequence has the sequence of SEQ ID NO: 15; rpsD wherein the complementary nucleic acid sequence has the sequence of SEQ ID NO: 17; and fnrS wherein the complementary nucleic acid sequence has the sequence of SEQ ID NO: 13.

51. The method of claim 48, wherein the bacterial infection is caused by one or more multi drug resistant bacteria and the composition comprises at least one antisense antibiotic oligomer targeting one or more antibiotic targets in the one or more multi drug resistant bacteria.

52. The method of claim 51, wherein the composition comprises a first antisense antibiotic oligomer that targets a first target and a second antisense antibiotic oligomer that targets a second target different from the first target.

53. The method of claim 52, wherein the first target and the second target are located on the same DNA sequence, RNA sequence, or mRNA sequence.

54. The method of claim 53, wherein the first target and the second target are located on different DNA sequences, RNA sequences, or mRNA sequences.

55. The method of claim 54, wherein the a first mRNA sequence and a second mRNA sequence each encode a unique protein associated with antibiotic resistance, a unique protein essential for bacterial homeostasis, or a first mRNA sequence encoding a protein associated with antibiotic resistance and a second mRNA sequence encoding a protein essential for bacterial homeostasis.

56. The method of claim 52, wherein the first target is located on a DNA sequence and the second target is located on an mRNA sequence.

57. The method of claim 51, wherein the at least one antisense antibiotic oligomer is selected from the group of: folC wherein the complementary nucleic acid sequence has the sequence of SEQ ID NO: 7; ffh wherein the complementary nucleic acid sequence has the sequence of SEQ ID NO: 9; lexA wherein the complementary nucleic acid sequence has the sequence of SEQ ID NO: 11; gyrB wherein the complementary nucleic acid sequence has the sequence of SEQ ID NO: 15; rpsD wherein the complementary nucleic acid sequence has the sequence of SEQ ID NO: 17; and fnrS wherein the complementary nucleic acid sequence has the sequence of SEQ ID NO: 13.

58. The method of claim 48, wherein the subject is selected from the group of: human; cattle; swine; poultry; goat; sheep; dog; cat; rodent; bird; and reptile.

59. A method for preventing emergence of antibiotic resistance in a subject in need thereof, comprising administering to the subject a pharmaceutically effective amount of a composition of claim 27 wherein the at least one antisense antibiotic oligomer targets at least one of a DNA sequence, RNA sequence, or mRNA sequence associated with antibiotic resistance.

60. The method of claim 59, wherein the subject is selected from the group of: human; cattle; swine; poultry; goat; sheep; dog; cat; rodent; bird; and reptile.

61. A method for identifying target genes involved in adaptive antibiotic resistance in bacteria comprising:
pretreating antibiotic-resistant bacteria with at least one antisense antibiotic oligomer of claim 1;
incubating the pretreated antibiotic-resistant bacteria with the at least one antisense antibiotic oligomer and an antibiotic to which the bacteria is resistant;
selecting one or more colonies of appearing after the incubation step;
determining the expression of two or more stress response genes;
determining a fold increase for each of the two or more stress response genes relative to a control wherein antibiotic-resistant bacteria were not pretreated with the composition; and
identifying a target gene involved in adaptive antibiotic resistance, wherein a fold increase of about 2 or more for a particular gene as determined fold increase determining step is indicative of a target gene involved in adaptive antibiotic resistance.

62. A method for developing an antibacterial antisense therapeutic comprising:
identifying at least one target gene involved in adaptive antibiotic resistance; and
designing an antisense oligomer complimentary to an mRNA sequence of the at least one target gene identified in step a), thereby developing an antibacterial antisense therapeutic.

63. The method of claim 62, wherein the identifying step comprises:
pretreating antibiotic-resistant bacteria with at least one antisense antibiotic oligomer of claim 1;
incubating the pretreated antibiotic-resistant bacteria with the at least one antisense antibiotic oligomer and an antibiotic to which the bacteria is resistant;
selecting one or more colonies of appearing after the incubation step;
determining the expression of two or more stress response genes;
determining a fold increase for each of the two or more stress response genes relative to a control wherein antibiotic-resistant bacteria were not pretreated with the composition; and
identifying a target gene involved in adaptive antibiotic resistance, wherein a fold increase of about 2 or more for a particular gene as determined by the fold increase determining step is indicative of a target gene involved in adaptive antibiotic resistance.

64. The method of claim 62, wherein the designing step is repeated for each identified target gene.


SEQUENCE LISTING (54 sequences)

SEQ ID NO: 1 - DNA, 12 bp, Artificial Sequence
  Description of Artificial Sequence Synthetic oligonucleotide
cctttttcaata

SEQ ID NO: 2 - DNA, 12 bp, Artificial Sequence
  Description of Artificial Sequence Synthetic peptide nucleic acid (PNA) sequence
cctttttcaata

SEQ ID NO: 3 - DNA, 12 bp, Artificial Sequence
  Description of Artificial Sequence Synthetic oligonucleotide
tactcatactct

SEQ ID NO: 4 - DNA, 12 bp, Artificial Sequence
  Description of Artificial Sequence Synthetic peptide nucleic acid (PNA) sequence
tactcatactct

SEQ ID NO: 5 - DNA, 12 bp, Artificial Sequence
  Description of Artificial Sequence Synthetic oligonucleotide
gaataagggcga

SEQ ID NO: 6 - DNA, 12 bp, Artificial Sequence
  Description of Artificial Sequence Synthetic peptide nucleic acid (PNA) sequence
gaataagggcga

SEQ ID NO: 7 - DNA, 12 bp, Artificial Sequence
  Description of Artificial Sequence Synthetic oligonucleotide
taatcatggtat

SEQ ID NO: 8 - DNA, 12 bp, Artificial Sequence
  Description of Artificial Sequence Synthetic peptide nucleic acid (PNA) sequence
taatcatggtat

SEQ ID NO: 9 - DNA, 12 bp, Artificial Sequence
  Description of Artificial Sequence Synthetic oligonucleotide
tcaaacattgtc

SEQ ID NO: 10 - DNA, 12 bp, Artificial Sequence
  Description of Artificial Sequence Synthetic peptide nucleic acid (PNA) sequence
tcaaacattgtc

SEQ ID NO: 11 - DNA, 12 bp, Artificial Sequence
  Description of Artificial Sequence Synthetic oligonucleotide
gctttcattccg

SEQ ID NO: 12 - DNA, 12 bp, Artificial Sequence
  Description of Artificial Sequence Synthetic peptide nucleic acid (PNA) sequence
gctttcattccg

SEQ ID NO: 13 - DNA, 12 bp, Artificial Sequence
  Description of Artificial Sequence Synthetic oligonucleotide
cacctgcaagag

SEQ ID NO: 14 - DNA, 12 bp, Artificial Sequence
  Description of Artificial Sequence Synthetic peptide nucleic acid (PNA) sequence
cacctgcaagag

SEQ ID NO: 15 - DNA, 12 bp, Artificial Sequence
  Description of Artificial Sequence Synthetic oligonucleotide
ttcgacatcaac

SEQ ID NO: 16 - DNA, 12 bp, Artificial Sequence
  Description of Artificial Sequence Synthetic peptide nucleic acid (PNA) sequence
ttcgacatcaac

SEQ ID NO: 17 - DNA, 12 bp, Artificial Sequence
  Description of Artificial Sequence Synthetic oligonucleotide
ttgccattttct

SEQ ID NO: 18 - DNA, 12 bp, Artificial Sequence
  Description of Artificial Sequence Synthetic peptide nucleic acid (PNA) sequence
ttgccattttct

SEQ ID NO: 19 - DNA, 20 bp, Artificial Sequence
  Description of Artificial Sequence Synthetic primer
aagcccttcttccagacgtg

SEQ ID NO: 20 - DNA, 20 bp, Artificial Sequence
  Description of Artificial Sequence Synthetic primer
aacggcaaagccaaagtgtc

SEQ ID NO: 21 - DNA, 20 bp, Artificial Sequence
  Description of Artificial Sequence Synthetic primer
gccttcctgtttttgctcac

SEQ ID NO: 22 - DNA, 20 bp, Artificial Sequence
  Description of Artificial Sequence Synthetic primer
ataataccgcgccacatagc

SEQ ID NO: 23 - DNA, 20 bp, Artificial Sequence
  Description of Artificial Sequence Synthetic primer
tggtaatgggcttctcgtcg

SEQ ID NO: 24 - DNA, 20 bp, Artificial Sequence
  Description of Artificial Sequence Synthetic primer
tggtttcgcctggaagtacc

SEQ ID NO: 25 - DNA, 20 bp, Artificial Sequence
  Description of Artificial Sequence Synthetic primer
ggccagtttgtgattacgcc

SEQ ID NO: 26 - DNA, 20 bp, Artificial Sequence
  Description of Artificial Sequence Synthetic primer
ctacgctcccacaaaatgcg

SEQ ID NO: 27 - DNA, 20 bp, Artificial Sequence
  Description of Artificial Sequence Synthetic primer
atccgttcctgaacgcactg

SEQ ID NO: 28 - DNA, 20 bp, Artificial Sequence
  Description of Artificial Sequence Synthetic primer
actgtgatgagaaaccgggc

SEQ ID NO: 29 - DNA, 20 bp, Artificial Sequence
  Description of Artificial Sequence Synthetic primer
gttaacggccaggcaacaag

SEQ ID NO: 30 - DNA, 20 bp, Artificial Sequence
  Description of Artificial Sequence Synthetic primer
tcaataacgcctttgcgtgc

SEQ ID NO: 31 - DNA, 20 bp, Artificial Sequence
  Description of Artificial Sequence Synthetic primer
aatcgcgcaaaagctgaagg

SEQ ID NO: 32 - DNA, 20 bp, Artificial Sequence
  Description of Artificial Sequence Synthetic primer
gcgattcgccctgcatattg

SEQ ID NO: 33 - DNA, 20 bp, Artificial Sequence
  Description of Artificial Sequence Synthetic primer
atggaacgtgagcaggacag

SEQ ID NO: 34 - DNA, 20 bp, Artificial Sequence
  Description of Artificial Sequence Synthetic primer
cagccagcgtttcagcatac

SEQ ID NO: 35 - DNA, 20 bp, Artificial Sequence
  Description of Artificial Sequence Synthetic primer
gatccagcgttgaccaagtg

SEQ ID NO: 36 - DNA, 22 bp, Artificial Sequence
  Description of Artificial Sequence Synthetic primer
cgccagataccattttgatgcg

SEQ ID NO: 37 - DNA, 20 bp, Artificial Sequence
  Description of Artificial Sequence Synthetic primer
agggcgtcacagatttccag

SEQ ID NO: 38 - DNA, 20 bp, Artificial Sequence
  Description of Artificial Sequence Synthetic primer
gtaaaaccacgctgacgctg

SEQ ID NO: 39 - DNA, 20 bp, Artificial Sequence
  Description of Artificial Sequence Synthetic primer
atcagcggcgtatcttccag

SEQ ID NO: 40 - DNA, 20 bp, Artificial Sequence
  Description of Artificial Sequence Synthetic primer
accgcttcgactctcaacag

SEQ ID NO: 41 - DNA, 20 bp, Artificial Sequence
  Description of Artificial Sequence Synthetic primer
aacggcgggcaatttttacc

SEQ ID NO: 42 - DNA, 20 bp, Artificial Sequence
  Description of Artificial Sequence Synthetic primer
aactgttatcgcagggagcc

SEQ ID NO: 43 - DNA, 20 bp, Artificial Sequence
  Description of Artificial Sequence Synthetic primer
tctgctgcgagacataaccc

SEQ ID NO: 44 - DNA, 20 bp, Artificial Sequence
  Description of Artificial Sequence Synthetic primer
acttgcaacgaatgttccgc

SEQ ID NO: 45 - DNA, 20 bp, Artificial Sequence
  Description of Artificial Sequence Synthetic primer
acgcactaccaccagtaacg

SEQ ID NO: 46 - DNA, 20 bp, Artificial Sequence
  Description of Artificial Sequence Synthetic primer
tttgtcttccgggaccagtg

SEQ ID NO: 47 - DNA, 20 bp, Artificial Sequence
  Description of Artificial Sequence Synthetic primer
attccgttctcggtggttcc

SEQ ID NO: 48 - DNA, 20 bp, Artificial Sequence
  Description of Artificial Sequence Synthetic primer
ccagcgtctgtttttctgcc

SEQ ID NO: 49 - protein, 10 aa, Artificial Sequence
  Description of Artificial Sequence Synthetic peptide
KFFKFFKFFK

SEQ ID NO: 50 - RNA, 136 nt, Artificial Sequence
  Description of Artificial Sequence Synthetic polynucleotide
ugcuucaauaauauugaaaaaggaagaguaugaguauucaacauuuccgugucggccuuauucccuuuuuugcggcauuuugccuuccuguuuuugcucacccagaaacgcuggugaaaguaaaagaugcugaaga

SEQ ID NO: 51 - RNA, 14 nt, Artificial Sequence
  Description of Artificial Sequence Synthetic oligonucleotide
uuaagcauugguaa

SEQ ID NO: 52 - DNA, 70 bp, Artificial Sequence
  Description of Artificial Sequence Synthetic oligonucleotide
  Description of Combined DNA/RNA Molecule Synthetic oligonucleotide
gauaaatgcuucaauaauauugaaaaaggaagaguaugaguauucaacauuuccgugucgcccuuauucc

SEQ ID NO: 53 - RNA, 27 nt, Artificial Sequence
  Description of Artificial Sequence Synthetic oligonucleotide
auauugaaaaaggaagaguaugaguau

SEQ ID NO: 54 - RNA, 14 nt, Artificial Sequence
  Description of Artificial Sequence Synthetic oligonucleotide
gucgcccuuauucc